(19) United States
(12) Patent Application Publication
(10) Pub. No.: US 2005/0123479 A1
(43) Pub. Date: Jun. 9, 2005

(54) Therapeutic properties of oils

(76) Inventor: Antonio Ferrante, Mount Osmond (AU)

Correspondence Address: O M Sam Zaghmout;BioIps, 8509 Kernon Court, Lorton, VA, 22079, US

(21) Appl. No.: 10/505,446

(22) PCT Filed: Mar. 3, 2003
(86) PCT No.: PCT/AU03/00266

(30) Foreign Application Priority Data
Mar. 1, 2002 (AU) PS 0821

(51) Int. Cl. A61K 35/12; A61K 31/225

(52) U.S. Cl. 424/9.8; 424/522; 514/548

(57) ABSTRACT

The present invention provides a novel scientific approach to determine whether a compound has anti-inflammatory activity. In particular, the novel assays allow the screening of compounds for the purposes of prophylactic and therapeutic use in treating or ameliorating the symptoms of T-cell, macrophage or neutrophil mediated diseases in mammals. In particular, the invention is based on the measurement of the capacity of a substance being an oil or fat, an alcoholic extract of an oil or fat, a biologically active component of an oil or a fat, or a preparation comprising an oil or fat, to suppress the activity of T-cells, macrophages or neutrophils in humans or animals in response to chemical and/or biological agents that activate these cell types. Measurements are made either in vivo (eg in mice) or in an in vitro preparation of human T-cells, macrophages or neutrophils, or a cell line derived therefrom. The substance is, in particular, emu oil or an ethanolic extract thereof. Therapeutic compositions and methods are also disclosed.

### BACKGROUND OF THE INVENTION

The immune system plays a critical role in the prevention of disease and the maintenance of health.

Diminished immune function, as occurs in the aged, in children under the age of two years, and in burns patients, as well as patients undergoing chemotherapy or transplantation, can increase the risk of disease.

On the other hand, inappropriate or excessive response of the immune system to infective agents or various stressors can result in tissue damage. Accordingly, autoimmune and allergic inflammatory diseases continue to be a major burden to the community. These diseases result from the “inappropriate” stimulation of leukocytes of the immune system, which include lymphocytes, macrophages and neutrophils. For example, chronic immune system activation can increase the risk of disease, eg arthritis, cystic fibrosis, inflammatory bowel disease, Crohn's disease, graft versus host disease, multiple sclerosis (MS), systemic sclerosis, allergic contact dermatitis, psoriasis and diabetes. The main approaches to treating these diseases are to depress the immunological reactions by inhibiting a variety of responses of leukocytes (1).

There are numerous reports showing that animal and plant fats and oils have therapeutic properties through their ability to modulate immune function; eg fish oils, flaxseed oil, linseed oil, borage oil, emu oil and evening primrose oil.

The Australian aboriginal practice of external application of emu oil for treating aches and pains has provided anecdotal evidence for the anti-inflammatory properties of this oil (2,3). However, conclusive scientific evidence for the in vivo efficacy of anti-inflammatory properties of emu oil is lacking, with only limited studies in experimental arthritis in rodents having been conducted thus far (4,5). It is well appreciated in the emu oil industry that the anti-inflammatory efficacy of different preparations of emu oil varies significantly. This variation can be so significant that it hampers the therapeutic use of this oil (6) and hence its commercial value. At the moment, no standard protocols are followed in the farming or source of emu, the part of the bird from which the oil is obtained, the method of preparation or storage of emu oil (7). In fact, there are conflicting data on the therapeutic efficacy of different emu and other oils and there appear to be at least two reasons for this.

Firstly, most animal fats and oils are complex mixtures with highly variable chemical compositions. The individual components almost certainly have different effects on immune function and may, in addition, inhibit the activities of other components or even synergise with each other.

Secondly, the immune system is made up of a number of different cell types, each with highly specific roles and not all of which respond in the same way to fats and oils. Optimum activity of an oil is therefore dependent on the condition being treated, as the cell types each have defined roles.

Furthermore, current scientific assays and tests on the efficacy of oils have presented conflicting results. The inability to quality control and standardise the oil for anti-inflammatory properties has posed a major limitation to the use of emu oil as a therapeutic agent. Variations in these factors can, in part, contribute to variations in the efficacy of the oil and have prevented its use in humans as a pharmaceutical agent, more particularly as a treatment for inflammatory diseases, conditions or responses.

A accurate assessment of the immunosuppressive activity prior to therapeutic use would greatly increase the consistency and reproducibility of treatment with a particular oil, as well as providing a means of increasing its therapeutic activity.

Unfortunately, the prior art is lacking in methods of assessing the likely therapeutic activity of an oil sample.

The present inventors have developed a method of measuring the intrinsic capacity of an oil to suppress the immune system of humans and animals. The method also allows the testing of the level of therapeutic activity of an oil, thereby enabling differentiation between oil samples of low and high therapeutic activity, and enabling oils to be graded for their therapeutic activity.

### SUMMARY OF THE INVENTION

According to one aspect, the present invention overcomes or reduces at least some of the above-mentioned problems by providing a novel scientific approach to accurately determine whether a compound has anti-inflammatory activity. In particular, the novel assays allow the screening of compounds for the purposes of prophylactic and therapeutic use in treating or ameliorating the symptoms of T-cell, macrophage or neutrophil mediated diseases in mammals.

In particular, the invention is based on the measurement of the capacity of an oil or fat, alcoholic extracts of an oil or fat, biologically active components of an oil or a fat, or preparations comprising oils or fats, to suppress the activity of T-cells, macrophages or neutrophils in humans or animals in response to chemical and/or biological agents that activate these cell types. Measurements are made either in mice (ie in vivo) or in human T-cells, macrophages or neutrophils isolated from blood. The method can be used to quantify the total T-cell, macrophage and/or neutrophil suppressive activities per unit mass or volume in any oil or fat and the degree of suppression of T-cell, macrophage or neutrophil responses by an oil or fat.

Using a model representative of a chronic inflammatory reaction (the delayed type hypersensitivity (DTH) reaction), emu oil was found to inhibit T lymphocytes and macrophage recruitment to the site of inflammation.

Emu oil was also found to significantly suppress the acute inflammatory response induced by Carrageenan reaction. Alcoholic, and in particular ethanolic, soluble fractions of emu oil were found to inhibit the ability of neutrophils to adhere to endothelial cells, but in particular were found to substantially suppress the chemotactic response of neutrophils.

The effects of emu oil and its ethanol soluble components on T-cell, macrophage and neutrophil chemotaxis and recruitment indicate that both emu oil and its ethanol soluble components are useful for treating acute and chronic inflammatory reactions.

After dissolving emu oil in ethanol, the soluble fraction of emu oil (containing primarily triglycerides) was found to have anti-inflammatory properties and contradicts the earlier belief that emu oil by itself does not have anti-inflammatory properties. The inventors have conclusively shown that the ethanol soluble fraction of the emu oil suppresses T-lymphocyte activity in that it suppresses both lymphoproliferation and also the production of pro-inflammatory and pro-DTH cytokines such as interleukin-2, lymphotoxin and interferon-γ. These activities of T-lymphocytes play fundamental roles in inflammation. Further fractionation of the ethanol soluble fraction showed that certain components contributed to anti-inflammatory activity, whilst others suppressed anti-inflammatory activity.

The inventors also found that the efficacy of the anti-inflammatory properties of the emu oil was dependent on the temperature at which the oil was rendered from emu fat. Activity was found with oils rendered at temperatures of 60° C. and 80° C., and ever better activity with oils rendered at 100° C. However, preparations prepared at 40° C. had minimal activity.

According to a first aspect of the invention, there is provided an assay system for testing samples of substances (such as emu oils and other oils) to assess, in a standardized manner, the anti-inflammatory activity of each sample, and to enable different samples to be graded in terms of anti-inflammatory activity (if any).

The assay system may involve administration of serially reducing amounts of the test substance (eg serially diluted in ethanol) to test animals (eg mice). Administration may be by injection (eg into the footpad), or be intraperitoneal, topical or oral administration.

In one embodiment of the invention, the assay system comprises assessing the anti-inflammatory activity of a compound or composition, herein referred to as the test substance, by
(i) injection of a suitable antigen into an appropriate body part (eg footpad) of a mammal, for example a mouse;
(ii) either injection of a predetermined amount of said test substance into the same body part, or topical application to said mammal of a predetermined amount of said substance;
(iii) measurement of the degree to which swelling which would otherwise result from injection of said antigen is reduced or alleviated, for example in either the footpad or the immune system organs (eg lymph nodes); and
(iv) comparing the activity of said test substance, as measured in step (iii), against the activity of a standard compound having known anti-inflammatory characteristics, the activity of said standard compound having been measured by this same assay system of steps (i) to (iii), and having been used to generate a grading system to compare the efficacy of various test substances.

The antigen may, for example, be Carrageenan or sheep red blood cells (SRBC), and the test substance may be an emu oil or other oil believed to have anti-inflammatory activity.

In step (i), it is preferred that the antigen is injected either intraperitoneally or into the footpad or ear of a mouse. In step (ii), it is preferred that the test substance is injected intraperitoneally or applied topically.

The measurement of step (iii) is preferably undertaken some time, and in particular about 24 hours, after injection of the test substance (step (ii)).

An alternative, in vitro assay system for testing a substance so as to assess, in a standardised manner, its anti-inflammatory activity comprises:
(i) measurement of the activity of an in vitro preparation of T-cells, macrophages or neutrophils, or a cell line derived therefrom;
(ii) addition of said substance to said preparation of T-cells, macrophages or neutrophils, or said cell line derived therefrom;
(iii) measurement of the change in activity of said preparation of T-cells, macrophages or neurophils, or said cell line derived therefrom, following addition of said substance in step (ii); and
(iv) comparing the change in activity (as measured in step (iii)) for said substance against the change in activity for a standard compound having known anti-inflammatory characteristics, the change in activity for the standard compound having been measured by this same assay system of steps (i) to (iii), and having been used to generate a grading system to compare the efficacy of various test substances.

This in vitro assay system may involve treating the preparation of T-lymphocytes, macrophages or neutrophils, or said cell line derived therefrom, with serially reducing amounts of the test substance, eg serially diluted in ethanol.

This assay system is a means for assessing the effect of the oil being tested on the cell (eg T-cell, macrophage or neutrophil) mediated immune response elicited by an antigen, and hence assessing its anti-inflammatory activity.

The following are examples of the types of in vitro assays which can be carried out, according to this assay system:
(a) using a preparation of T lymphocytes, and measuring lymphoproliferation;
(b) using a preparation of T lymphocytes, and measuring their production of cytokines, such as interleukin-2 (IL-2), tumor necrosis factors (eg TNF α and lymphotoxin (TNF β)) and interferon γ (IFN-γ);
(c) using a preparation of neutrophils, and measuring their chemotatic activity; and
(d) using a preparation of neutrophils, and measuring their adherence to endothelial cells.

T-cells play a major role in the tissue damage in various diseases, largely through their production of cytokines. Cytokines (such as TNF α and IL-2) produced by T-cells are believed to contribute to the tissue damage resulting from abnormal immune function.

The use of therapeutic agents, preferably agents that are not toxic, to inhibit the production of cytokines by T-cells would be particularly useful in the treatment of tissue damage, particularly those mediated by T-cells.

Prior art agents used to treat T-cell mediated diseases are either toxic or have considerable systemic effects.

The present inventors have developed a method of treating or preventing tissue damage using (in particular) emu oil, a non-toxic material produced from the adipose tissue of emus. The inventors have developed a method of increasing the activity of the emu oil used for this purpose, thereby ensuring reliability and consistency of the product and, moreover, have found that permeants (substances used to increase the movement of chemical substances through the skin) are not required for activity. The inventors have also found that an alcoholic extract of emu oil so produced is also effective in treating T-cell mediated diseases.

The invention also relies on the discovery that emu oil, and alcoholic extracts of emu and other oils, are able to suppress the activity of T-cells, being cell types that contribute to the tissue damage in a variety of human diseases. The invention involves the use of emu and other oils, as well as extracts thereof, to treat these different disease states by preventing or reducing the damage caused by T-cells. The use of emu oil has a further advantage in that it can also reduce the tissue damage caused by another important immune cell type, the neutrophil.

Therefore, according to a second aspect of the invention, there is provided a composition comprising emu oil, or a biologically active extract or component thereof, optionally together with a carrier vehicle, for treating or ameliorating the symptoms of T-cell mediated diseases or conditions or neutrophil mediated diseases or conditions in mammals. Examples of the diseases or conditions include immune complex disease, renal disease, nephritis, arthritis (eg rheumatoid arthritis or septic arthritis), glomerulitis, vasculitis, gout, urticaria, angioedema, cardiovascular disease, systemic lupus erythematosus, breast pain/premenstrual syndrome, asthma, neurological disease, attention deficit disorder (ADD), psoriasis, retinal disease, acne, sepsis, granulomatosis, inflammation, reperfusion injury, cystic fibrosis, adult respiratory distress syndrome, thermogenesis, diabetes, inflammatory bowel disease, Crohn's disease, multiple sclerosis (MS), systemic sclerosis, osteoarthritis, atopic dermatitis, allergic contact dermatitis, graft rejection (graft versus host disease) or transplantation.

The composition can be in the form of an oral, injectable or topical composition. The biologically active extracts or components include at least one of the following: triglyceride fractions or triglyceride fraction components, sterol fractions or sterol fraction components, phenolic fractions or phenolic fraction components, alkali-stable fractions or alkali-stable fraction components, organic solvent extracts (eg of emu oil) or components thereof. In the preferred form, the organic solvent is ethanol.

According to a third aspect of the invention, there is provided a method of treating or ameliorating the symptoms of T-cell mediated diseases or conditions or neutrophil mediated diseases or conditions in mammals, the method comprising administering an effective dose of a composition comprising emu oil, or a biologically active extract or component thereof (eg as exemplified above).

The composition can be administered orally, parenterally (eg by injection) or topically.

It is preferred that said effective dose of said composition be administered after or just before a T-cell mediated disease or condition, neutrophil mediated disease or condition or inflammation reaction has occurred.

In a fourth aspect of the invention, an alcohol (such as ethanol), is used to extract compounds having anti-inflammatory activity from the emu oil or other biologically active oil or fat. Alternative organic solvents which would perform the same function of solubilising and extracting effective compounds from the oil would be apparent to persons skilled in the art.

Although emu oil is specifically exemplified, it is to be understood by those skilled in the art that the assays, methods and compositions of the present invention can be applied to any substance or oil of which emu oil is but one example. Other suitable oils are, for example, other animal oils; plant oils, such as tea tree oil, flaxseed oil, linseed oil, borage oil or evening primrose oil; fish oils; and algal, microbial and fungal oils.

According to a fifth aspect of the invention, there is provided a method of preparing or rendering emu oil for therapeutic use in a mammal, including the step of heating the emu oil, or the tissue from which the emu oil is derived, to a temperature of at least 40° C.

As used throughout the present specification and claims, the term “biologically active” refers to the capacity to elicit an anti-inflammatory response.

### DETAILED DESCRIPTION OF THE INVENTION

The active ingredient(s) in emu oil that is (are) responsible for the reported anti-inflammatory activity has (have) not been identified. Emu oil is composed mainly of triglycerides that contain varying amounts of fatty acids (Table 1). The limited available data on the composition of emu oil suggest that the clear oil can vary markedly in terms of anti-oxidants (carotenoids, flavonoids), skin permeation-enhancing factors and α-linolenic acid (18:3ω3) (from 0-20%) (4) content. The finding that the oil is not rich in ω3 fatty acids makes it unlikely that the anti-inflammatory effect of the oil is related to ω3 fatty acids, which are widely perceived as having anti-inflammatory actions. A previous study has reported, as unpublished results, that the efficacy of emu oil as an anti-inflammatory agent did not correlate with ω3 fatty acid content (0.2-19.7%) of the oil (4).

TABLE 1
Fatty acid composition of emu oil

| COMPONENT | AMOUNT |
|---|---|
| Oleic acid (18:1ω9) | 47-58% |
| Palmitic acid (16:0) | 19-24% |
| Stearic acid (18:0) | 8-11% |
| Linoleic acid (18:2ω6) | 5.5-17% |
| Hexadecenoic acid (16:1ω7) | 3-6% |

A combination of Thin Layer Chromatography (TLC), Gas Chromatography (GC) and Gas Chromatography-Mass Spectroscopy (GC-MS) analyses demonstrated the presence of a wide range of fatty acids, sterols and phenols in emu oil preparations. From the TLC, it was evident that triacylglycerol is the major component and needs to be considered as one of the anti-inflammatory components of the oil since previous studies have shown that fatty acids can inhibit inflammation.

In terms of its phenolic content, Makin emu oil was found to have 25 μmol/l which is about 20 fold less than the level of phenols in olive oil. Thus, it is unlikely that this is the active anti-inflammatory element of emu oil since olive oil has been reported not to have anti-inflammatory properties (7) (also, our unpublished observations).

Sterol analyses revealed that emu oil was similar to tuna oil but substantially different from olive oil, with cholesterol making up the major component of the emu oil sterols. The role of these substances in the anti-inflammatory properties of emu oil was not evaluated.

The fatty acid composition of the oil was analysed independently by three different groups using GC-MS, MS and GC. From these studies, it was found that the major fatty acids are oleic (around 50%), palmitic (around 20%), stearic (around 10%), linoleic (around 10%) and palmitoleic (around 5%). These could be taken as the main fatty acid components of Australian emu oils. The composition of oils prepared from emus in different geographical locations and probably prepared in different ways were not distinguishable based on the fatty acid content analyses.

Extensive studies using a standard emu oil (Makin) demonstrated that, when administered to mice, the oil consistently caused depression of chronic and acute inflammation. For chronic inflammation, a standard delayed type hypersensitivity reaction (DTH), which is induced and elicited by SRBC antigens, was used. The reaction was measured by monitoring the amount of hind footpad swelling as a result of an antigen challenge. Makin emu oil significantly inhibited the elicitation of this inflammatory response. Since the cells involved are predominantly T lymphocytes and macrophages, an effect, either directly or indirectly, on the accumulation of these cell types must have been caused by the administration of emu oil. The effects of emu oil were not restricted to chronic inflammation, since it was just as effective in depressing carrageenan-induced inflammation, considered to be a model for testing acute inflammation and which primarily involves neutrophil accumulation at the injected site.

Using the chronic inflammatory model of DTH, the effects of different preparations of emu oil on this response were examined in an effort to explain the reasons for variability in the efficacy of the different preparations. Of the samples of emu oil examined, Makin emu oil was the most effective. Toowoomba and Little Meadow showed some anti-inflammatory activity, less was seen with emu oil A2-100G and none with emu oil G53. This could not be explained on the fatty acid composition of the emu oil samples, since these were essentially similar (Tables 5 and 6 on pages 29 to 30).

Examination of the characteristics of the depressive effects of emu oil on inflammation showed that the oil was most effective when given close to or just after the antigen challenge. This was shown by the fact that the efficacy of the emu oil was greatest when the oil was given 1 h before, rather than 5 h before, challenge. A similar effect was shown using the carrageenan-induced inflammation model. It was also found that, when the emu oil treatment was delayed to 3 h after the elicitation of the inflammatory response, the efficacy of the emu oil was significantly more effective than treatments given 1 h before challenge. Firstly, this suggests that the oil acts quite rapidly on components of the immune system; secondly, this shows that inflammation can be controlled using suitably prepared emu oil even after an individual begins to experience inflammation.

Rendering temperature was found to govern the efficacy and/or type of oils produced since emu oil extracted at 40° C. was found to be less active than when it was extracted at 60° C., 80° C. or 100° C. No evidence was found in terms of fatty acid composition by GC analysis between the oils produced at the latter three different temperatures since these were very similar in content, including the levels of linoleic acid (18:2ω6) (see, for example, Table 11 on page 40).

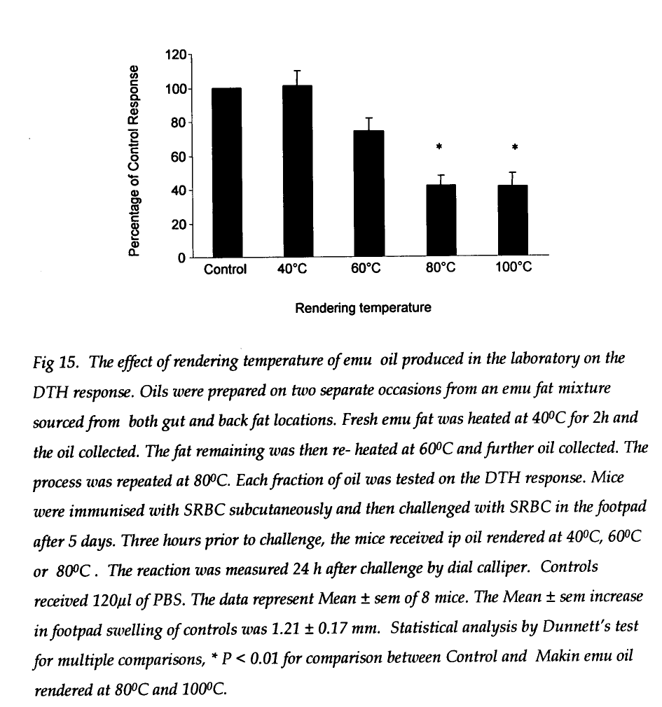

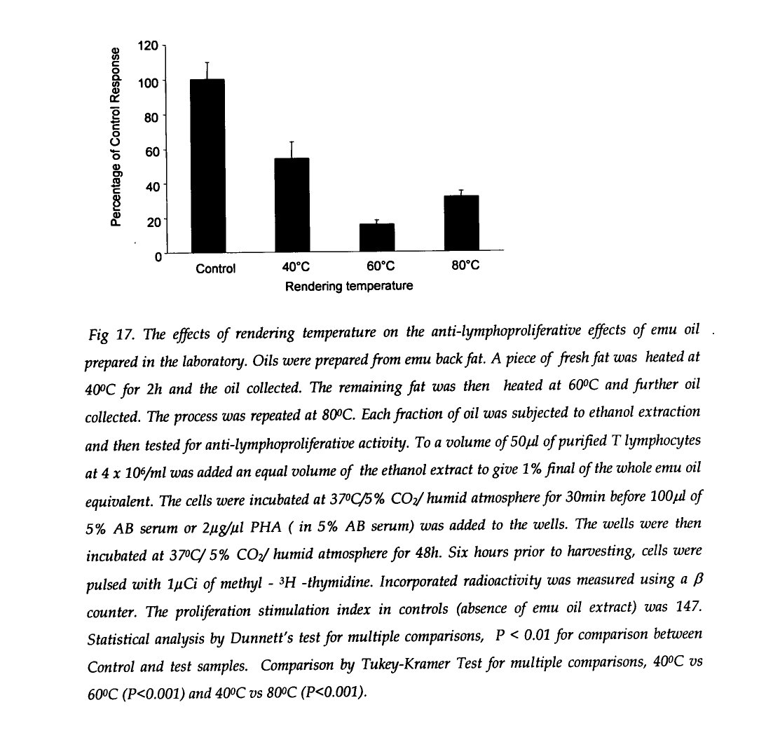

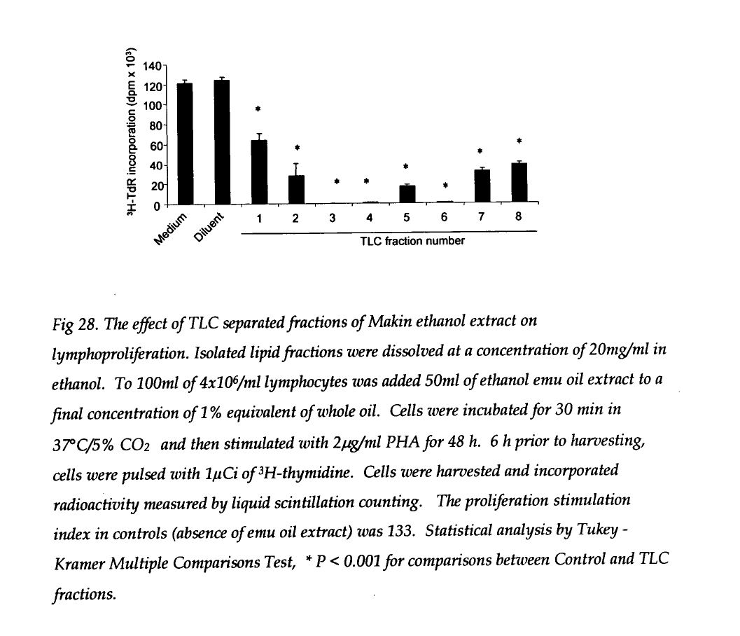

To identify the components in emu oil responsible for the anti-inflammatory effects, the emu oil was added directly to cultured lymphocytes and neutrophils in order to see if the activities of these leukocytes would be altered. The studies were unsuccessful because of the solubility problem of the oil. To overcome this problem, the oil was solubilised in ethanol and, following fractionation, the components having anti-inflammatory activity were identified (see FIG. 28). The solubilised fraction had significant anti T lymphocyte activity. Since T lymphocytes are the major cell which mediate the DTH reaction and chronic inflammation, these results show that emu oil is able to suppress DTH activity. Chemical analysis of the ethanol fraction by GC did not reveal any enrichment of a particular fatty acid, although there was, however, a slight increase in the proportion of 18:2ω6. Thus, the ethanol soluble fraction may be a source from which the active components can be used to treat inflammation. Interestingly, the anti T cell activity in terms of inhibition of lymphoproliferation in the emu oil preparations rendered at 40° C., 60° C. and 80° C. correlated with their in vivo activity with inhibition of DTH activity. The inventors have shown that, in both instances, rendering temperature of 60° C.<T≦100° C. produces more efficacious oils than rendering at 40° C. (FIG. 15 cf FIG. 17).

Further evidence for an effect on the T cell responses was shown by examining the effects of the ethanol soluble emu oil fraction on the cytokine products produced by activated T lymphocytes, IL-2, lymphotoxin, TNF β and IFN-γ. Production of these cytokines was inhibited by pre-treating T lymphocytes with the solubilised emu oil fraction. The effects were extended to production of TNF by monocytes via LPS stimulation. However, it was evident that the T cell production of cytokines was more sensitive to emu oil than TNF production by monocytes, showing a preferential effect of the ethanol soluble emu oil fraction for T lymphocyte responses, suggesting the T cell as a major target for emu oil therapy.

The solubilised fraction of Makin emu oil was found to inhibit both chemotactic migration as well as adhesion of neutrophils to endothelial cells. Both of these properties are key functions necessary for infiltration of neutrophils to sites of inflammation. Neutrophil adherence was also affected when endothelial cells were pre-treated with the solubilised fraction. The combination of the effects of the solubilised fraction on the neutrophils and endothelial cells would inhibit adherence of leukocytes to endothelial cells in vivo. While the effect on neutrophils is not relevant to DTH, it is highly relevant to carrageenan induced or acute inflammation, where the neutrophil is thought to be a key player (9).

The emu oil ethanol soluble fraction was found to be rich in free fatty acids (see Table 13 on page 45). Thus, one of the effects on T lymphocytes could involve fatty acids such as 18:2ω6. The inventors' investigations established that serum fatty acid binding proteins such as albumin can decrease the activity of free fatty acids by binding to them. Further investigations were conducted as to whether or not serum could abrogate the effects of a Makin emu oil ethanol extract, which had been rendered at 40° C. The addition of serum was found to block most of the anti-T cell activity of this oil fraction and this would explain the discrepancies and variations in efficacy of emu oils to treat inflammation.

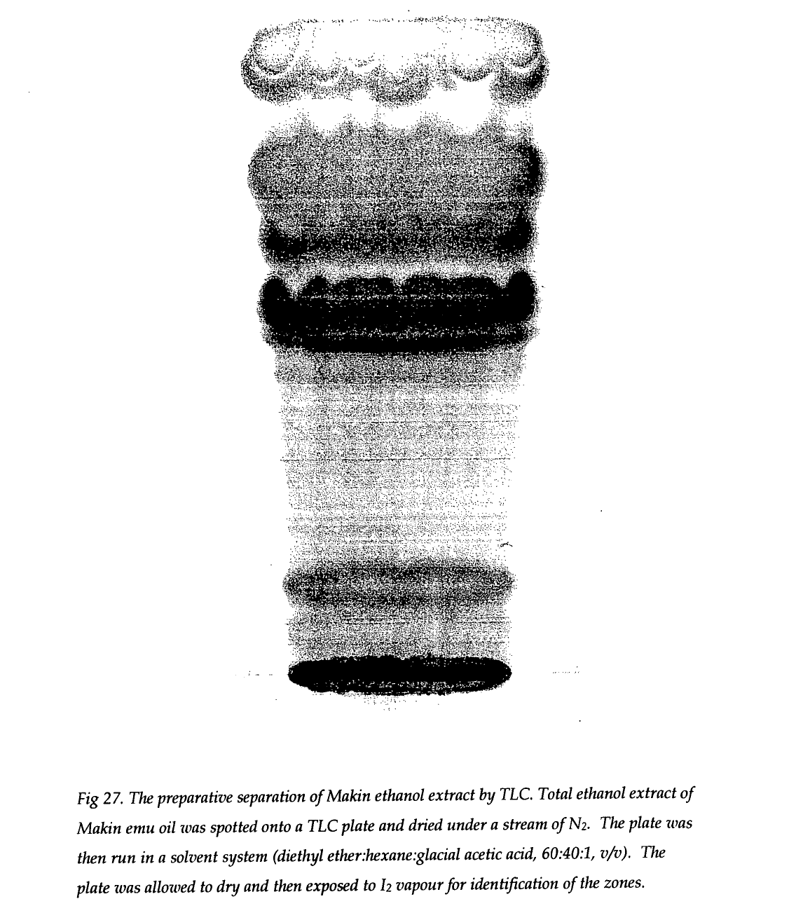

On TLC separation of the ethanol soluble fraction (see FIG. 27), several distinct bands were seen and at least one corresponded to the migration of the 18:2ω6 which was shown to be responsible for the majority of anti-T cell activity. However, other fractions were also active, suggesting that several emu oil components might be responsible.

The data from the experimental section below have revealed avenues which could be used to standardise emu oil, particularly for its anti-inflammatory activity. The results indicate that mice may be used as models of testing systems for chronic (DTH) and acute (carrageenan) inflammatory diseases. These represent simple systems in which inflammation can be readily quantified. To decrease variability, an ip route rather than topical emu oil administration is used. It has been established that the efficacy of an emu oil preparation may be determined by establishing the extent to which the preparation can be diluted before anti-inflammatory activity is lost. In this system of standardisation, an established, active emu oil can be used as a standard against which other emu oils may be tested. A criterion for accepting or rejecting emu oil preparations can then be established for the industry. The standard can be based on the optimal rendering conditions, as well as storage of oils, feed for emus, breed of emu etc (Table 2). Oil prepared at 100° C. was found to have the highest anti-inflammatory activity, whilst oil prepared at 40° C. had minimal activity.

Furthermore, the inventors found that the anti-inflammatory activity of emu oil was strongest when administered after inflammation had occurred. Also, the inventors found that administration of the emu oil 1 h prior to inflammation has better anti-inflammatory efficacy than if the oil is administered 3 h prior to inflammation.

TABLE 2
PREPARATION OF EMU OIL (potential causes of variability)

Collection of fat
Age of animal
Diet
Genetics
Sex
Length of time after death of the animal
Storage conditions of collected fat
Lipase/phospholipase/lipoxygenase activity
Non-enzymic oxidation
Rendering
Temperature of rendering
Type of container used
Amount of water
Surface area
Length of rendering time
Filtration
Temperature of filtration
Type of filter
Water in the filtrate
Metal content
Protein content
Variable crystallisation
Possible products formed during the processing of emu fat
Oxidation products of fatty acids
Free fatty acids
Lysophospholipids
Conjugated linoleic acid
Trans isomers
Diglycerides
Monoglycerides
Oxidation products of cholesterol

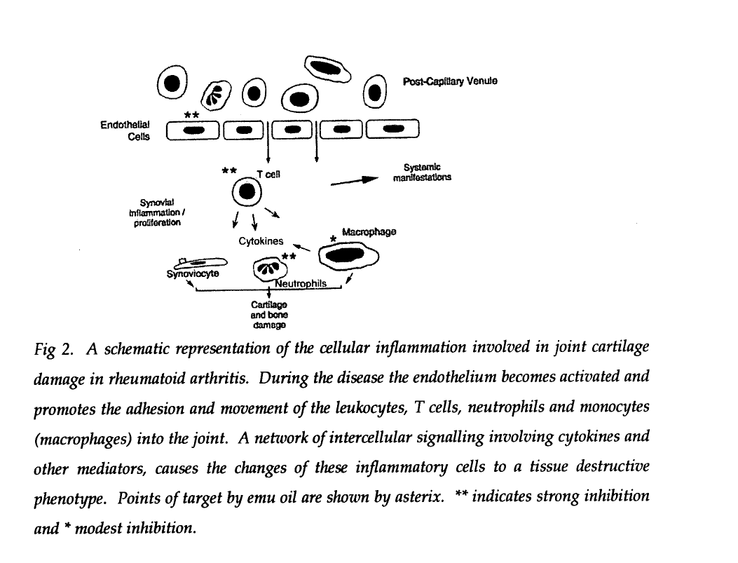

It is preferable to extend the testing by conducting in vitro assays to support the data from the in vivo chronic and acute inflammation reactions. This is particularly important before the oils can be commercially used. Thus, effect on T lymphocyte and monocyte function for chronic, and neutrophil function for acute, inflammation can be employed. A model is illustrated in FIG. 2.

Both for the DTH and carrageenan inflammatory response, a relationship can then be established for the amount of oil versus the degree of inhibition of inflammation.

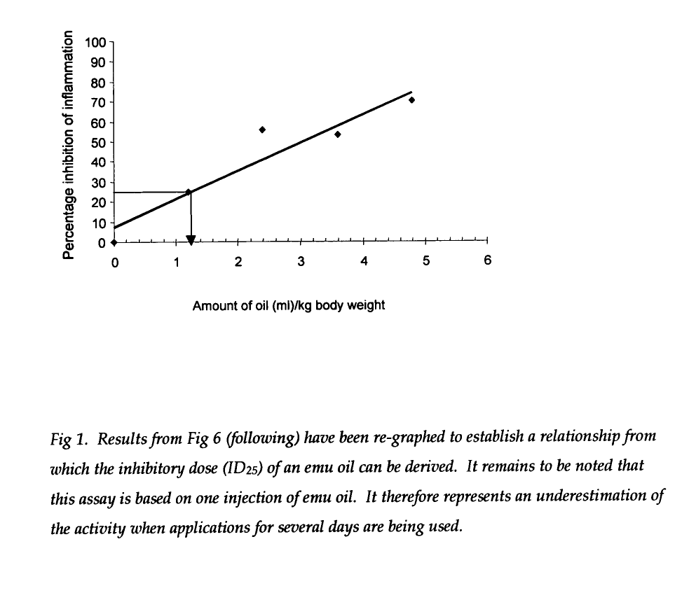

From the graph of FIG. 1, the emu oil concentration required to achieve 25% inhibition (ID₂₅) of the inflammatory responses can be deduced. From this value, the anti-inflammatory power of the oil can be determined. The values may be computed for both acute and chronic inflammation, where they may be different.

The above anti-inflammatory efficacy values can be corroborated by data using the ethanol soluble fraction of the oil, examining an effect on T lymphocyte function and neutrophil function. Two useful parameters are lymphoproliferation for T lymphocytes and chemotaxis for neutrophils for chronic and acute inflammation respectively. Similar ID₂₅ and maximal inhibition values based on these parameters can be computed as discussed above.

Based on the effects of emu oil on T lymphocyte and macrophage responses, as well as neutrophil responses, the therapeutic potential is apparent for diseases/conditions summarised in Table 3. The targets in the treatment of these inflammatory diseases are outlined, specifically those which are critical and are targeted by emu oil. The targets of emu oil have been further expanded in FIG. 2, which shows the events which lead to joint damage in rheumatoid arthritis. The T cells and macrophages, as well as neutrophils, are targeted and either prevented from migrating into the tissue and/or prevented from being activated to generate tissue destructive mediating cytokines.

TABLE 3
Therapeutic target for emu oil and the respective disease

| | TARGETS RELEVANT |
|---|---|
| CONDITION/DISEASE | TO EMU OIL THERAPY |
| Cardiovascular diseases | Endothelial cells, macrophages |
| Rheumatoid arthritis | T cells, macrophages and |
| | neutrophils |
| Atopic dermatitis | T cells, interferon γ |
| Inflammatory bowel disease | T cells, macrophages, neutrophils |
| Systemic lupus erythematosus | T cells and macrophages |
| Asthma | T cells, macrophages, neutrophils, |
| | cytokines |
| Cystic fibrosis | Macrophages and neutrophils |
| Breast pain/premenstrual syndrome | Oedema |
| Transplantation | T cells, cytokines |
| Neurological diseases | T cells, macrophages |
| Psoriasis | T lymphocytes, interferon γ |
| Diabetes renal, retinal and | Endothelial cells, macrophages, |
| cardiovascular complications | neutrophils |
| Gout | Neutrophils |
| Acute respiratory distress syndrome | Neutrophils, cytokines |
| Acne | Neutrophils, cytokines |
| Septic arthritis | Neutrophils, cytokines |
| Reperfusion injury | Neutrophils, cytokines |

In summary, the data herein has shown the complexity of the composition of emu oil, in which the fatty acid content was studied in detail. There are no major differences in the levels of the various fatty acid species in distinctly different preparations, in terms of geography, feed, rendering and storage. Nevertheless, there is a marked difference in the ability to depress inflammation. Using a freshly prepared standardised emu oil preparation (Makin), the anti-inflammatory properties of emu oil were tested, in chronic and acute in vivo and in vitro inflammation models. Some evidence points to at least some of the activity being due to an unsaturated fatty acid, 18:2ω6, but the study has demonstrated the difficulty in trying to identify what gives rise to the anti-inflammatory properties. Be that as it may, the inflammatory models developed can be used to standardise the anti-inflammatory activity of emu oil, which would seem to be a prerequisite for developing a viable industry, using quality-controlled Australian oils.

Materials and Methods

Emu Oils

Details of the emu oils used in the study are outlined in Table 4. The emu oils were kept frozen at −20° C. in aliquots.

TABLE 4
Description of the different preparations of emu oils used in the present study

| | | | Age of birds at | |
|---|---|---|---|---|
| | Rendering Process | Age of Oil | slaughter | Feed |
| Makin | Back fat @ 40 C. | 2 months | 1-15 months old | Feed lot mix |
| G53 | Gut fat @ 40 C. | 4 years | 1-<3 years old | Grainfed & |
| | | | | range |
| A2-100G | Gut fat @ 40 C. | 4 years | 5-<3 years old | Grainfed & |
| | | | | range |
| Toowoomba | Back fat @ 40 C. | 4 years | 2-<3 years old | Grainfed & |
| | | | | range |
| Little Meadow | Gut & back fat @ | 2 years | Unknown | Emu pellets & |
| | 104 C. | | | range |
| Gut Fat A | Gut fat rendering | 1 year | 1-15 months old | Farmed: Green |
| | temperature | | | clovers, weeds & |
| | unknown | | | grasses, milled |
| Back Fat A | Back fat rendering | 1 year | 1-15 months old | barley, triticale, |
| | temperature | | | wheat & luceme, |
| | unknown | | | canola oil |
| Gut Fat B | Gut fat rendering | 1 year | 1-15 months old | |
| Back Fat B | Back fat rendering | 1 year | 1-15 months old | |
| | temperature | | | |
| | unknown | | | |
| Commercial | Unknown | Unknown | Unknown | Unknown |

1. Preparation of Ethanol Soluble/Insoluble Fractions

To obtain the ethanol soluble fraction, 2 ml of emu oil was mixed with 1 ml of ethanol, centrifuged at 2,500 g/3 min/4° C. and the upper phase collected. The extraction procedure was repeated three times on the lower phase. These ethanol soluble fractions were pooled, centrifuged and dried under N₂ gas stream. Eventually, stocks of 2 ml volume were made for experiments; also, the ethanol insoluble fraction (EIF) remaining was retained as a rich source of triglyceride.

2. Fatty Acid Analyses

2.1 Thin Layer Chromatography

Up to 1 mg emu oil in 20-40 μl chloroform-methanol (4:1) was applied as a 1 cm band to the edge of a TLC plate. Linoleic acid (18:2) was applied as a standard in a 0.5 cm band to one side of the test sample. The chromatogram was developed in hexane-ether-acetic acid (80:20:1) and dried in the fume hood. The zones were viewed by exposure to 12 vapour or sprayed lightly with 18N H₂SO₄ and charred at 150° C. Larger amounts of Makin emu oil were dissolved in chloroform-methanol (4:1), and aliquots of the solution (equivalent to 5 mg of oil) were applied as a 6-7 cm band to a silica thin layer plate. An equivalent amount of olive oil dissolved in the same solvent mixture was applied to the plate as a 6-7 cm band and served as a control. An unesterified fatty acid standard was applied to the edges of the plate. A chromatogram was developed in hexane-ether-acetic acid (80:20:1) and, after drying, the plate was exposed to iodine vapour.

2.2 NMR Analysis

This was performed by Dr N. Trout, Flinders University. To a dry flask (5 ml) was added 100-120 mg of the thawed emu oil (shaken thoroughly), which was dissolved in dry toluene (1-1.2 ml). To this was added a freshly prepared solution of sodium methoxide (75 mg Na in methanol (2 ml)) under N₂. The resulting mixture was placed under reflux for ninety minutes, before cooling and adding acetic acid (100 μl) and water (2.5 ml). The white mixture was extracted with hexane twice before the layers were dried over Na₂SO₄, filtered and the volatiles removed in vacuo. ¹³C and ¹H NMR measurements were recorded on a Varian Gemini FT 300 MHz multinuclear spectrometer, operating at 75.46 MHz and 300.75 MHz respectively. All samples were dissolved in deuterated chloroform, using the central peak (77.0 ppm) for ¹³C and CHCl₃ (7.26 ppm) for ¹H NMR referencing. To a NMR tube was added 75-100 mg of the emu oil followed by deuterated CDCl₃ (0.8 ml). The resulting solution was analysed by NMR. After one hour of pulsing, the spectrum was printed to show all the signals indicative of a triglyceride.

2.3 GC Analyses

Child Health Research Institute (Dr. R. Gibson/Mr. M. Neumann). One drop of emu oil was methylated in 5 ml of 1% sulphuric acid (36N) in methanol for 2 hours at 70° C. After cooling, the resulting methyl esters were extracted into 2 ml of n-heptane and transferred to vials containing anhydrous sodium sulphate as the dehydrating agent. Emu oil fatty acid methyl esters were separated and quantified using a Hewlett-Packard 6890 gas chromatograph equipped with a 50 m capillary column (0.33 mm ID) coated with BPX-70 (0.25 μm film thickness—SGE Pty Ltd, Victoria, Australia). The injector temperature was set at 250° C. and the flame ionisation detector at 300° C. The initial oven temperature was 140° C. and was programmed to rise to 220° C. at 5° C. per minute. Helium was used as the carrier gas at a velocity of 35 cm per second. Fatty acid methyl esters were identified based on retention time to authentic lipid standards from Nuchek Prep Inc (Elysian, Minn.).

RMIT (Prof A. Sinclair/Ms. K. Murphy): Samples were analysed in duplicate. An aliquot of whole lipid was taken and dried using a stream of nitrogen. Samples were hydrolysed to free fatty acids using 7.9% KOH (Univar, AJAX chemicals, Australia) in methanol (Merck, Germany). Samples were cooled and converted to fatty acid methyl esters (FAME) using 20% boron trifluoride (BF₃) in methanol complex (Merck, Germany). Gas Chromatographic analyses were performed using a Shimadzu GC 17A GC fitted with a flame ionisation detector (FID). FAME were analysed using a BPX-70 50 m cross-linked 70% Cyanopropyl Polysilphenylene-siloxane capillary column with an ID of 0.32 mm and 0.25 μm film thickness. Samples were injected at 125° C. and held for 1.0 minute. The oven temperature was set to increase by 5° C./min to 170° C. and held for 4 minutes, then by 0.5° C./min to 175° C. and 4° C./min to a final temperature of 220° C. which was held for 3 minutes. The injector and detectors were maintained at 260° C. and helium was used as the carrier gas. Peak area and concentrations were quantified on an IBM compatible computer using Shimadzu software (Japan).

2.4 GC-MS

GC-MS analysis was performed on a Varian Saturn 4D instrument with a J&W DB 5% phenylmethylpolysiloxane column (30 m×0.25 mm id).

2.5 MS

Women's and Children's Hospital (Dr. D. Johnson): 1 mg of emu oil was treated with benzene/methanol/acetyl chloride at 100° C. for 90 min. After cooling, the neutralised solution was extracted with hexane and samples of the extract were injected into a Perkin Elmer Turbomass Mass Spectrometer.

3. Sterol Analysis

These experiments were carried out by Ms K Murphy from the laboratory of Professor A. Sinclair at the Royal Melbourne Institute of Technology. Sterol-enriched fractions were obtained from two emu oil samples (Makin and G53) by alkaline saponification with 5% KOH in methanol/water (80:20, v/v), followed by extraction with 2 ml of hexane:chloroform (4:1, v/v) three times. The sterols were then converted to their corresponding trimethylsilyl ethers (OTMSi) with BSTFA (N,O-Bis(trimethylsilyl)trifluoroacetamide) for 15 minutes at 70° C. Gas chromatographic analyses were performed using a Shimadzu GC 17A GC fitted with a FID and a BPX-5 50 m (5% Phenyl Polysilphenylene-siloxane) with an ID of 0.32 mm and 0.25 μm film thickness. Samples were injected at 200° C. and held for 1 minute. The oven temperature was set to increase by 20° C./min to 340° C. and held for 30 minutes. The injector and detector were maintained at 280° C. and helium was the carrier gas. Peak area and concentrations were quantified on an IBM compatible computer using Shimadzu software (Japan).

4. Analysis of Phenolics

The analysis of phenolics in a sample of Makin emu oil, in two other emu oils, and in a number of other fats and oils was carried out in the laboratory of Dr P. Hayball at the University of South Australia. The total phenolic content was determined using a modification of the Folin-Ciocalteau method and results were expressed as gallic acid equivalents.

5. Inflammation Models

5.1 Delayed type hypersensitivity (DTH) reaction: The DTH response was induced in 12 week old female BALB/c mice (Animal Resource Centre, Perth) as described previously (8). Briefly, mice were injected with sheep red blood cells (100 μl of 10% haematocrit) (SRBC; Sigma Chemical Co.). After 5 days, the animals were challenged intradermally in the right hind footpad with SRBC (25 μl of 40%-haematocrit) or into the left footpad with diluent (25 μl). The DTH response was determined 24 h post challenge and was calculated by comparing the thickness between the diluent vs SRBC injected footpads. Footpad thickness was measured with a dial calliper.

5.2 Carrageenan-induced paw reaction: Carrageenan-induced paw reaction was induced as described previously (9,10). Mice were inoculated with carrageenan (1 ml/kg of a 1% solution) (Type IV; Sigma Chemical Co.) into the right hind paw. The reaction was assessed by measuring hind paw thickness at the indicated times.

6. Leukocyte Separation

Mononuclear leukocytes (MNL) and neutrophils were prepared by the rapid single-step separation method (11). Briefly, whole blood was layered onto Hypaque-Ficoll medium of density 1.114 and then centrifuged at 400 g/30 min. After centrifugation, the leukocytes resolve into two distinct bands. The upper band contained MNL and the lower band the neutrophils.

7. Lymphocyte Proliferation

Lymphocyte proliferation was measured by a semi-automated microtechnique (12). Human mononuclear cells (2×10⁵) were seeded into u-bottomed wells of a micro-titre plate (50 μl) and treated with 500 of the ethanol emu oil fraction. After 30 min incubation, 2 μg/μl PHA was added to stimulate the T lymphocytes. The cells were incubated for 72 h at 37° C. in an atmosphere of 5% CO₂-air and high humidity. At 6 h prior to harvest, the cultures were pulsed with 1 μCi of ³H-TdR. The cells were harvested and the amount of radioactivity incorporated measured in a liquid scintillation counter.

8. Cytokine Production

Production of IL-2, IFN-γ and lymphotoxin (TNF β) by T lymphocytes was measured in MNL stimulated with PHA as described for lymphocyte proliferation. The supernatants from cell cultures were collected and the amount of cytokine measured by ELISA using cytokine specific monoclonal antibodies as described previously (13).

Production of the cytokine TNF α by monocytes was measured in MNL stimulated with LPS. Briefly, 2×10⁵ MNL in a 100 μl volume was added to flat bottomed wells of a microtitre plate and then the cells were stimulated by adding 100 μl of 200 ng/ml bacterial lipopolysaccharide (LPS). After incubation at 37° C./48 h, the supernatant was collected for TNF a measurement, using an ELISA and TNF a specific monoclonal antibody as described previously (13).

9. Neutrophil Adhesion

9.1 To Plasma Coated Surfaces

Adhesion was assessed by the ability of neutrophils treated with emu oil extract to bind to plasma-coated plates after stimulation with TNF α. Plates which had been coated with autologous plasma (1:10), washed and dried received 50 μl neutrophils (5×10⁶/ml) which were treated for 30 mins at 37° C./5% CO₂. The neutrophils were stimulated with TNF α (10³ units/ml) for 30 mins at 37° C./5% CO₂, washed with HBSS, then stained with 100 μl Rose Bengal (0.25% w/v PBS) at room temperature. Non-adherent cells were removed by washing with HBSS, and then 200 μl ethanol: PBS (1:1) was added and development proceeded at room temperature for 30 mins before reading on a plate reader at 570 nm.

9.2 Neutrophil Adherence to Human Umbilical Vein Endothelial Cells (HUVEC).

HUVECs were isolated from umbilical cords stored at 4° C. after delivery, as previously described (15) but with 0.2% (w/v) gelatin (Cytosystems) to coat all tissue culture flasks and plates, 0.07% (w/v) collagenase (from Clostridium histolyticum, type II, Worthington) to digest the interior of the umbilical vein, and a culture medium consisting of RPM1640 (ICN-Flow) containing 40 mmol/l TES, 15 mmol/l D-glucose, 80 U/ml penicillin (Flow), 80 μg/ml streptomycin (Flow), and 3.2 mmol/l L-glutamine, which was brought to 260 to 300 mOsm/l before the addition of 20% (v/v) pooled, heat-inactivated (56° C., 30 minutes) human group AB serum. Endothelial cells were identified by their characteristic contact-inhibited cobblestone morphology and positive staining for factor VIII-related antigen using peroxidase-conjugated anti-rabbit IgG to human von Willebrand factor (Dako) and 3,3′-diaminobenzidine.

Confluent cultures were subcultured after 2 to 5 minutes exposure to trypsin (0.05% [v/v], Flow)-EDTA (0.02% [w/v]). For experimental use, second-passage cells were plated at 2×10⁶ cells per well per 0.2 ml culture medium in 96-well culture plates. The HUVECs were treated with the emu oil ethanol soluble fraction and then with TNF-α, the monolayers were washed once with RPMI 1640, before incubation for 30 minutes at 37° C. in the absence or presence of 5×10⁵ neutrophils in E-SFM (final volume, 100 μl). Nonadherent cells were removed by gentle aspiration, and the wells were washed twice with HBSS containing 0.1% (w/v) μM phorbol myristate acetate (PMA) to stimulate the cells' BSA before staining with rose bengal. After release of the dye with 50% ethanol, the absorbance (570 nm) of each well was determined with an ELISA plate reader. Test and blank wells were performed in triplicate. Results were calculated after subtraction of the mean blank value (without leukocytes) from each test value (plus leukocytes) (15).

10. Neutrophil Chemotaxis

Chemotaxis was measured by the migration under agarose method as previously described (16). Six millilitres of 1% molten agarose in medium 199 containing 5% fetal calf serum were poured into petri dishes. After the agarose solidified, sets of three holes/wells were punched in the agarose layer. Plates with these sets of three wells were used to measure leukocyte migration in a chemotaxis gradient, with 50 μl of 1×10⁻⁷M fMLP, 5 μl of neutrophils (2.5×10⁵) and 5 μl of medium 199 being added to the inner, centre and outer wells respectively. Two well sets were used to measure random migration, cells being added to one well and medium to the other. The plates were incubated at 37° C. and the distance of cell migration measured directly under a phase-contrast microscope after 90 min. The approximate migration distances of neutrophils in assays conducted in our laboratory were 2.2 mm and 0.7 mm in the presence and absence of fMLP, respectively.

11. Results

11.1 Chemical Composition of Emu Oil

Analyses of emu oil were conducted at a number of different centres to enable a better assessment of the various constituents of the oil. Fatty acid analyses of emu oils were made at the Women's and Children's Hospital in Adelaide, Flinders University, and at the Royal Melbourne Institute of Technology (RMIT), Victoria. Analysis of phenolic content of the oil was conducted at the University of South Australia and sterol analysis at RMIT. The results are all presented and, in some cases, comparisons between the same oils from analyses made at different centres are outlined.

11.2 Fatty Acid Composition of Emu Oils

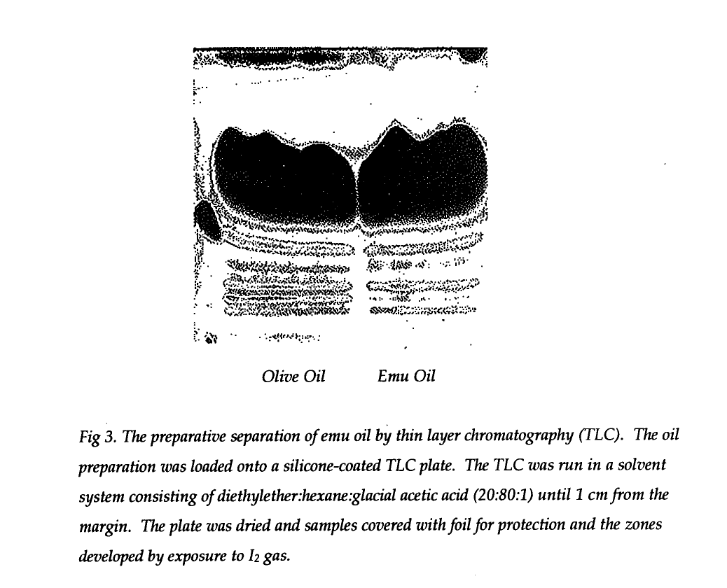

Examination by thin layer chromatographic analysis of emu oil showed that the major component of emu oil is triacylglycerol. However, smaller amounts (around 1-2%) of at least 7 other minor components were detected (FIG. 3). Three of these were tentatively identified as unesterified fatty acids, diacylglycerol, and sterols.

The identity of the other components was not established. Some of these had a similar chromatographic mobility to compounds present in olive oil. These experiments indicate that emu oil is a more complex mixture than previously believed. As many of the minor components in olive oil are thought to contribute to its properties, particularly its health benefits, it is likely that the minor components in emu oil may also have a similar effect. Apart from a band in olive oil running near the solvent and tentatively identified as the hydrocarbon, squalene, the chromotagraphic profile of emu oil did not appear very different from olive oil, although it is likely that there are some components that are unique to each oil.

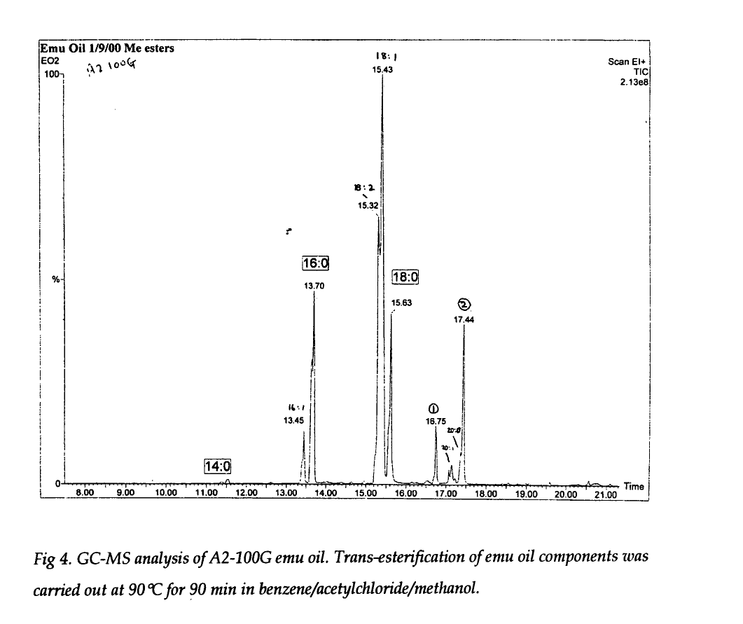

The fatty acid composition of the nine emu oils analysed by GC-MS at Flinders University by Dr Neil Trout (organic chemist) is shown in Table 5. The predominant fatty acid was oleic acid (18:1ω9). This ranged from 49% to 58% of the fatty acids in the nine oils. The next most prominent fatty acid was palmitic acid (16:0), which ranged from 19-22%. Other prominent fatty acids were stearic acid (18:0) ranging from 9-11%, linoleic acid (18:2ω6) ranging from 5.5-17% and hexadecenoic acid (16:1ω7) ranging from 3-6%. A typical GC-MS trace of the fatty acid analyses is seen in FIG. 4.

TABLE 5
GC-MS Analysis of nine preparations of emu oil. GC-MS analyses were
performed on a Varian Saturn 4D instrument with a J&W DB5/phenylmethyl
polysiloxane column (30 m × 0.25 mm).

| | fatty acid | | | | | | | | |
|---|---|---|---|---|---|---|---|---|---|
| Emu oil | 14:0 | 14:1 | 16:0 | 16:1 | 17:0 | 18:0 | 18:1 | 18:2 | 20:0/20:1 |
| Little Meadow | Trace | trace | 20.18 | 5.79 | trace | 8.84 | 50.12 | 10.40 | trace |
| | | | | | | | 4.65 | | trace |
| | | | | | | | trace | | |
| Toowoomba | Trace | trace | 20.17 | 3.63 | trace | 11.60 | 49.12 | 9.04 | trace |
| | | | | | | | 3.23 | | trace |
| | | | | | | | trace | | |
| Gut Fat A | Trace | trace | 21.35 | 5.22 | trace | 10.45 | 48.87 | 9.21 | trace |
| | | | | | | | 4.89 | | trace |
| | | | | | | | trace | | |
| G53 | Trace | trace | 20.13 | 3.88 | trace | 11.65 | 58.33 | 2.79 | trace |
| | | | | | | | 2.70 | | trace |
| | | | | | | | trace | | |
| A2-100G | Trace | trace | 19.48 | 3.98 | trace | 11.64 | 54.28 | 5.45 | trace |
| | | | | | | | 4.60 | | trace |
| | | | | | | | trace | | |
| Makin | Trace | trace | 18.92 | 3.53 | trace | 11.04 | 49.60 | 14 | trace |
| | | | | | | | 2.91 | | trace |
| | | | | | | | trace | | |
| Back Fat A | Trace | trace | 22.25 | 5.27 | trace | 10.92 | 49.31 | 8.38 | trace |
| | | | | | | | 3.86 | | trace |
| | | | | | | | trace | | |
| Duncan 170M | Trace | trace | 19.65 | 3.50 | trace | 10.13 | 52.32 | 11.13 | trace |
| | | | | | | | 3.26 | | trace |
| | | | | | | | trace | | |
| Duncan 176M | trace | trace | 19.20 | 2.85 | trace | 8.83 | 49.78 | 16.70 | trace |
| | | | | | | | 2.70 | | trace |
| | | | | | | | trace | | |

Analyses of these oils were also undertaken in Dr Bob Gibson's laboratory at Flinders University (Table 6). Nine emu oil samples were analysed by this method. Examination of GC traces showed that the fatty acid composition was much more complex than had been suspected, with upwards of two dozen different fatty acids identified. Many of these components were only present in trace amounts (<0.1%). Emu oil contains mainly straight chain even numbered carbon chain fatty acids, the major saturates being palmitic (16:0) and stearic (18:0) acids, with only small amounts of shorter (14:0) and longer (20:0 and 22:0) chain saturates (Table 6).

TABLE 6
GC Analyses of emu oil fatty acids

| Fatty Acid | Gut FatB | GutFatA | A2-100G | 53G | Back FatA | Back FatB | Little Meadow | Toowomba | Makin |
|---|---|---|---|---|---|---|---|---|---|
| 8:0 | | | | | | | | | |
| 9:0 | | | | | | | | | |
| 10:0 | | | | | | | | | |
| 11:0 | | | | | | | | | |
| 12:0 | 0.03 | 0.03 | 0.03 | 0.03 | 0.03 | 0.02 | 0.03 | 0.04 | 0.03 |
| 13:0 | | | | | | | | | |
| 14:0 | 0.30 | 0.34 | 0.25 | 0.28 | 0.33 | 0.30 | 0.33 | 0.26 | 0.42 |
| 15:0 | 0.03 | 0.03 | 0.03 | 0.04 | 0.03 | 0.03 | 0.03 | 0.03 | 0.07 |
| dma 16:0 | | | | | | | | | |
| 16:0 | 23.88 | 23.18 | 20.00 | 19.27 | 23.91 | 23.80 | 23.71 | 20.36 | 20.54 |
| 17:0 | 0.09 | 0.10 | 0.10 | 0.14 | 0.10 | 0.09 | 0.09 | 0.12 | 0.22 |
| dma 18:0 | | | | | | | | | |
| 18:0 | 10.85 | 8.47 | 9.42 | 11.52 | 8.75 | 9.94 | 8.28 | 10.73 | 11.14 |
| 20:0 | 0.19 | 0.21 | 0.16 | 0.18 | 0.18 | 0.18 | 0.12 | 0.17 | 0.21 |
| 22:0 | 0.03 | 0.02 | 0.02 | | 0.03 | 0.02 | | | 0.03 |
| 24:0 | | | | | | | | | |
| Total Sats | 35.40 | 32.38 | 30.01 | 31.47 | 33.35 | 34.39 | 32.58 | 31.71 | 32.67 |
| Trans 16:1 | | | | | 0.23 | | 0.07 | | 0.03 |
| Trans 18:1ω9 | 0.24 | 0.24 | 0.30 | 0.39 | 0.05 | 0.24 | 0.91 | 0.37 | 0.32 |
| Trans 18:1ω7 | 0.06 | | | | | | | | 0.19 |
| Trans 18:2 | | 0.01 | | | | 0.02 | | | 0.04 |
| Totals Trans | 0.29 | 0.26 | 0.30 | 0.39 | 0.28 | 0.24 | 1.00 | 0.37 | 0.58 |
| 11:1 | | | | | | | | | |
| 12:1 | | | | | | | | | |
| 13:1 | | | | | | | | | |
| 14:1 | 0.07 | 0.11 | 0.07 | 0.07 | 0.11 | 0.09 | 0.11 | 0.06 | 0.09 |
| 15:1 | | | | | | | | | |
| 16:1ω9 | 0.10 | 0.11 | 0.13 | 0.12 | 0.10 | 0.10 | 0.18 | 0.13 | 0.15 |
| 16:1ω7 | 3.94 | 4.97 | 3.57 | 3.08 | 5.23 | 4.58 | 5.33 | 3.22 | 2.95 |
| 17:1 | | | | | | | | | |
| 18:1ω9 | 48.17 | 48.82 | 52.99 | 49.71 | 47.94 | 48.42 | 47.48 | 49.57 | 47.88 |
| 18:1ω7 | 2.32 | 2.60 | 2.39 | 2.15 | 2.55 | 2.27 | 2.72 | 2.05 | 2.68 |
| 19:1 | | 0.02 | 0.02 | | | | | | 0.05 |
| 20:1ω11 | 0.05 | 0.05 | 0.09 | 0.06 | 0.05 | 0.05 | 0.06 | 0.08 | 0.06 |
| 20:1ω9 | 0.47 | 0.45 | 0.40 | 0.48 | 0.46 | 0.45 | 0.29 | 0.45 | 0.41 |
| 22:1ω11 | | | | | | | | | |
| 22:1ω9 | 0.07 | 0.05 | 0.02 | 0.04 | 0.06 | 0.06 | 0.03 | 0.04 | 0.02 |
| 24:1ω9 | | | | | | | | | |
| Total Monos | 55.19 | 57.18 | 59.65 | 55.70 | 56.50 | 56.02 | 58.18 | 55.56 | 53.41 |
| 18:2ω9 | 0.04 | 0.04 | 0.02 | | 0.04 | 0.04 | 0.04 | | 0.02 |
| 20:2ω9 | | | 0.03 | | | | | 0.05 | 0.03 |
| 20:3ω9 | | | 0.03 | | | | 0.03 | 0.04 | 0.03 |
| Totalω9 | 48.84 | 49.47 | 53.61 | 50.35 | 48.60 | 49.08 | 48.04 | 50.27 | 49.26 |
| Totalω7 | 6.26 | 7.57 | 5.95 | 5.21 | 7.78 | 6.85 | 8.05 | 5.27 | 4.78 |
| 9.11 18:2 cLA | 0.05 | 0.01 | 0.05 | 0.05 | 0.05 | 0.05 | 0.03 | 0.04 | 0.06 |
| 10.12 18:12 cLA | | | | | | | | | |
| 18:2ω6 | 7.81 | 8.65 | 9.26 | 11.26 | 8.32 | 7.92 | 9.18 | 11.59 | 11.95 |
| 18:3ω6 | 0.04 | 0.04 | 0.02 | | 0.04 | 0.04 | 0.02 | | 0.04 |
| 20:2ω6 | 0.05 | 0.05 | 0.07 | 0.10 | 0.06 | 0.05 | 0.05 | 0.08 | 0.10 |
| 20:3ω6 | | | | | | | | | 0.02 |
| 20:4ω6 | 0.03 | 0.04 | 0.06 | 0.06 | 0.04 | 0.03 | 0.04 | 0.06 | 0.09 |
| 22:2ω6 | | 0.00 | | | | | | | |
| 22:4ω6 | | | 0.03 | | | | | 0.03 | 0.04 |
| 22:5ω6 | | | | | | | | | |
| Totalω6 | 7.92 | 8.79 | 9.44 | 11.42 | 8.47 | 8.05 | 9.30 | 11.76 | 12.24 |
| 16:2ω3 | | 0.02 | 0.03 | 0.03 | 0.02 | | 0.03 | | 0.08 |
| 18:3ω3 | 1.12 | 1.29 | 0.41 | 0.91 | 1.26 | 1.18 | 0.80 | 0.43 | 0.82 |
| 18:4ω3 | | | | | | | | | |
| 20:3ω3 | | | | | | | | | |
| 20:5ω3 | | | | | | | | | |
| 22:5ω3 | | 0.02 | 0.02 | 0.04 | 0.03 | 0.03 | | | 0.04 |
| 22:6ω3 | | | | | | | | | |
| Totalω3 | 1.12 | 1.34 | 0.46 | 0.98 | 1.31 | 1.21 | 0.82 | 0.43 | 0.94 |

The ranges for the predominant fatty acids were 18:1ω9 (47-53%), 16:0 (20-24%), 18:0 (8-11%), 18:2ω6 (8-12%) and 16:1ω7 (3-5%). Again, the greatest variability was seen in 18:2ω6 and 18:0. The main monoenoic acid was oleic acid (18:1ω9). Traces of shorter (16:1ω9) and longer chain (20:1ω9, 22:1ω9) monoenoic acids were detected. ω7 series monoenoic fatty acids were also present, the main one being 16:1ω7, which was present in significant amounts (around 3%). Only traces of odd numbered carbon chain fatty acids were detected. The main polyunsaturated fatty acid was linoleic acid (18:2ω6). Traces of other ω6 series polyunsaturated fatty acids were present, and included gamma linolenic (18:3ω6), arachidonic (20:4ω6), and docosatetraenoic (22:4ω) acids. ω3 Polyunsaturated fatty acids were minor components, the main one being alpha linolenic acid (18:3ω3), with only traces of 16, 20, and 22 carbon compounds. Conjugated linoleic acid (the 9, 11 isomer) was also detected, but only in very small amounts (<0.1%).

Fatty acid analysis was also carried out by Ms K. Murphy in the laboratory of Professor Andrew Sinclair at RMIT. Approximately 21 individual fatty acids were identified in the emu oils (Table 7). The dominant fatty acid class was the monounsaturated fatty acids (approximately 54-57%), followed by the saturated fatty acids (31-34%). Omega-6 fatty acids were the dominant polyunsaturated fatty acids identified, ranging from 8-12%, while omega-3 fatty acids were present at less than 2% of total PUFA.

Oleic acid (18:10ω9) was the dominant fatty acid in the emu oils (Table 7), ranging from 48.2% in the G53 emu oil to 49.2% in the Makin emu oil. Palmitic acid (16:0) was the next most dominant fatty acid (approximately 19-23%), followed by stearic acid (18:0) (10-11%), linoleic acid (18:20ω6) (8-12%) and hexadecenoic acid (cis16:1ω7) (3-4%). DHA predominated in the tuna oil, followed by 16:0, 18:1 ω9, 18:0, EPA, and cis 16:1ω7. Olive oil was predominantly 18:1ω9 (78%), with a smaller percentage of 16:0 (11%) and 18:0 (3%).

TABLE 7
GC Analysis of fatty acids of emu, tuna and olive oils at RMIT

| | Oil | | | | |
|---|---|---|---|---|---|
| Fatty acid | Emu (Makin) | Emu (53G) | Emu (KM) | Tuna | Olive |
| 12:0 | 0 | 0.42 | 0.14 | 0.01 | 0 |
| 12:1 | 0 | 0 | 0 | 0.70 | 0 |
| 14:0 | 0.49 | 0.29 | 0.14 | 2.59 | 0 |
| 14:1 | 0.06 | 0.43 | 0 | 0.85 | 0 |
| 15:0 | 0 | 0.01 | 0 | 0.77 | 0 |
| 16:0 | 21.94 | 18.82 | 23.33 | 17.07 | 11.09 |
| 16:1ω7t | 0.12 | 0.15 | 0 | 0.28 | 0.05 |
| 16:1ω7c | 3.13 | 3.00 | 3.61 | 3.31 | 0.66 |
| 17:0 | 0.12 | 0.22 | 0.04 | 1.85 | 0.08 |
| 17:1 | 0.06 | 0.03 | 0 | 0.86 | 0.03 |
| 18:0 | 11.32 | 11.0 | 9.53 | 6.27 | 2.87 |
| 18:1ω9 | 49.2 | 48.24 | 51.32 | 13.04 | 77.84 |
| 18:1ω7 | 1.77 | 2.09 | 2.42 | 2.17 | 1.66 |
| 18:2ω6 | 11.90 | 10.31 | 8.18 | 1.88 | 5.88 |
| 18:3ω3 | 0.75 | 0.89 | 1.61 | 0.54 | 0.16 |
| 18:4ω3 | 0 | 0 | 0 | 0.87 | 0 |
| 20:0 | 0.03 | 0.22 | 0.01 | 0 | 0.02 |
| 20:1ω11 | 0.07 | 0.53 | 0.07 | 0.60 | 0.01 |
| 20:1ω9 | 0 | 0 | 0 | 1.55 | 0 |
| 20:1ω7 | 0 | 0 | 0 | 0.12 | 0 |
| 20:2ω6 | 0.01 | 0.55 | 0 | 0.31 | 0 |
| 20:4ω6 | 0 | 0.17 | 0 | 2.55 | 0 |
| 20:3ω3 | 0 | 0 | 0 | 0.12 | 0 |
| 20:4ω3 | 0 | 0 | 0 | 0.60 | 0 |
| 20:5ω3 | 0 | 0 | 0 | 6.01 | 0 |
| 22:4ω6 | 0 | 0.62 | 0 | 1.06 | 0 |
| 22:5ω6 | 0 | 0 | 0 | 0.4 | 0 |
| 24:0 | 0 | 0.01 | 0 | 1.72 | 0 |
| 22:5ω3 | 0 | 0 | 0 | 1.14 | 0 |
| 22:6ω3 | 0 | 0 | 0 | 23.46 | 0 |

All figures are percent of total fatty acids present in the oil.

Examination of the GC/Mass spectrometric analysis of emu oil fatty acids by Dr. D. Johnson at the Women's and Children's Hospital confirmed that the main fatty acid components of emu oil were 14:0, 16:1, 16:0, 18:1, 18:2, 18:0, 20:0 and 20:1 (see FIG. 4). However, two other components, labelled as peaks 1 and 2, were also detected. These were not present in analyses carried out by two other laboratories. Neither peak was positively identified as a fatty acid, even though the 74 mass ion, indicative of fatty acid esters, was detected in both and was particularly prominent in peak 2. Based on a comparison of the peak heights as compared to other fatty acid peaks, peak 2 constituted around 34% of the total fatty acids in one of the emu oil samples analysed (Makin) and 6-7% in the other (A2-100G).

To explore the possibility that these two components were hydroxy fatty acids, samples of emu oil were hydrolysed with benzene/methanol/1% sulphuric acid at 100° C. for 2 hours. After extraction into hexane, samples of the hydrolysate were chromatographed on a TLC plate in hexane-ether-acetic acid (80:20:1) and the zones were detected by exposure to iodine vapour. Although under these conditions there had been almost complete hydrolysis of the emu oil, there was no evidence for the presence of hydroxy fatty acids. The only components detected were normal (unhydroxylated) fatty acid esters together with small amounts of alkali-stable lipids. One other possibility is that peaks 1 and 2 were formed by acetylation of diacylglycerols. Most animal and plant fats, including emu oil, contain small amounts of diacylglycerol generally formed by the breakdown of triacylglycerols. This possibility has not been investigated further.

11.3 Sterol Analysis

Approximately thirty sterols were present in the emu oils and tuna oil, while 28 sterols were present in the olive oil (Table 8). Of those, 15 sterols of the emu oils and 19 of the olive oil could not be identified with gas chromatography not linked to a mass spectrometer. Data has been presented as percentage of total sterols. Cholesterol was the major component of the sterol fraction of both Adelaide emu oil samples. It comprised 70% of the Makin and 55% of the G53 emu oil sterols respectively. A further 14 sterols were identified. The only other component present in significant amounts was 4, 23, 24-trimethyl-5α-cholest-22E-en-3β-ol (3.7 and 7.1%).

TABLE 8
Sterol analyses of emu, tuna and olive oil

| | Sample Oil (% of total sterols) | | | | |
|---|---|---|---|---|---|
| Sterol | Emu (Makin) | Emu (53G) | Emu KM | Tuna oil | Olive oil |
| Total unidentified peaks | 18 | 33 | 34 | 10 | 64 |
| 5α-cholestane | 0.5 | 0.3 | 0.2 | 0.5 | 1.1 |
| 24-nordehydrocholesterol | 1 | 0.5 | 0.1 | 3.1 | 1.0 |
| C26 sterol | 0.6 | 0.1 | 2.3 | 0 | 3.2 |
| Patinosterol | 0.6 | 0.1 | 0.1 | 0 | 1.1 |
| Trans-22-dehydrocholesterol | 0.6 | 0.1 | 0 | 0.9 | 0 |
| Cholesterol | 70 | 55 | 43 | 85 | 5 |
| Cholestanol | 1.5 | 1.1 | 0.3 | 0 | 0 |
| Desmosterol | 0.9 | 0.6 | 1.2 | 0 | 0 |
| Brassicasterol | 0.9 | 0.1 | 1.7 | 0 | 0 |
| 24-methylenecholesterol | 0.1 | 0 | 2.9 | 0 | 0.8 |
| 24-methycholesterol | 0.2 | 0.2 | 1.5 | 0 | 1.5 |
| Stigmasterol | 0.9 | 0.7 | 1.9 | 0 | 0 |
| β-sitosterol | 0.7 | 1.3 | 0.8 | 0 | 21 |
| Isofucosterol | 0.2 | 0.6 | 0 | 0.1 | 0 |
| 4,23,24-Trimethyl-5α-cholest- | 3.7 | 7.1 | 10.0 | 0 | 1 |
| 22E-en-3β-ol | | | | | |

All figures are percent of total sterol present in the oil.

A number of the other components, such as sitosterol, brassicasterol, and sitosterol, are plant sterols and therefore probably derived from the diet. A further 15 components, many of which are believed to be sterols, were also detected but they were not identified. These data provide further evidence for the complexity of emu oil and for the variability of its composition. The presence of plant sterols indicates that the concentration and composition of the minor components may be affected by diet.

Other sterols present in the Makin and G53 emu oils were an unidentified (UI) sterol eluting before cholesterol (5 and 13% respectively), an UI sterol eluting before 4,23,24-trimethyl-5α-cholest-22E-en-3β-ol (5 and 5% respectively), and cholestanol (2 and 1% respectively). The unidentified peaks were present in all samples tested and cannot be identified until gas chromatography with mass spectrometry is applied.

There were also traces of several additional sterols, including 5∝-cholestane, 24-nordehydrocholesterol, C26 sterol, patinosterol, trans-22-dehydrocholesterol, desmosterol, brassicasterol, 24-methylenecholesterol, 24-methylcholesterol, stigmasterol, β-sitosterol and isofucosterol (all ≦1%). β-sitosterol was the major sterol in the olive oil sample (21%). The peak identified as cholesterol (5%) in the olive oil sample is unlikely to be cholesterol. There is a possibility that it could be a long chain alcohol (28:0) which runs very close to cholesterol.

Tuna oil was comprised mainly of cholesterol (85%).

11.4 Polyphenol Analysis

The highest concentration of phenolics was found in olive oil, with values as high as 708 μmoles per litre (Table 9). Levels were very low in a number of other plant oils (sunflower, canola, and soya bean oils). The Makin emu oil had levels of phenolics that were comparable to those detected in castor and peanut oils (25.0 vs 21.7 and 25.0 and 27.1 and 30.0 mol per litre) (Table 9). As phenolics are normally found in plants, it is likely that the emu oil phenolics are derived from dietary sources. The total phenolic fraction of olive oil and other dietary oils normally comprises a mixture of simple and complex phenols. Although the emu oil phenolics were not identified, it is likely that they include a mixture of compounds. Their presence is a further indication of the complexity of emu oil. In view of their powerful antioxidant properties, and their ability to modulate the activity of immune cells (17), it is possible that they contribute to anti-inflammatory activity of emu oil, either directly or synergistically with other components present in the oil.

TABLE 9
Phenolic content in a range of plant and animal oils/fats.

| SAMPLE | Phenol concentration (μmol/l) |
|---|---|
| Canola Oil (No Frills) | 0.0 |
| Liquid Paraffin BP | 0.0 |
| Water | 0.0 |
| Sunflower Oil (Sunbeam) | 1.4 |
| Canola Oil (No Frills) | 1.4 |
| Water | 1.7 |
| Sunflower Oil (Sunbeam) | 5.7 |
| Liquid Paraffin BP | 8.3 |
| Soya Bean Oil | 8.6 |
| Soya Bean Oil | 8.6 |
| Ghee | 15.7 |
| Emu Oil (Emu Fire) | 13.3 |
| Ghee | 15.7 |
| Emu Oil (Emu Fire) | 18.3 |
| Castor Oil BP | 21.7 |
| Castor Oil BP | 25.0 |
| Peanut Oil | 27.1 |
| Peanut Oil | 30.0 |
| Olive Oil (J. Laforgia, Young Trees 2000) | 690.0 |
| Olive Oil (J. Laforgia, Young Trees 2000) | 708.6 |
| Makin Emu Oil | 25.0 |

12. Anti-Inflammatory Properties of Emu Oil
12.1 The Effect of Emu Oil on the Chronic Inflammatory Reaction

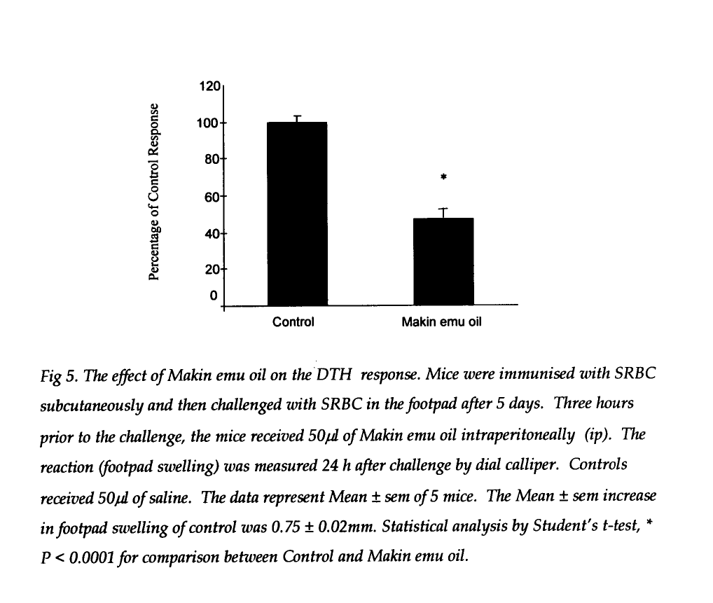

In these experiments, the Makin emu oil preparation was primarily used, as this had been prepared under “guided” conditions. The chronic inflammatory response was measured by the delayed type hypersensitivity reaction. This reaction is initiated by an antigen and elicited following antigen challenge at various sites. The response is characteristic of sensitised T lymphocytes, which mobilize and accumulate at the antigen challenge site. Such cells then cause the non-specific accumulation of other lymphocytes and a large infiltration of macrophages. This represents a significant model of the reactions seen in inflammatory diseases where tissue damage occurs. In these investigations, we used sheep red blood cells (SRBC) as the antigen for the delayed type hypersensitivity response. Mice were primed with SRBC subcutaneously and after 5 days challenged in the footpad with SRBC and the amount of swelling measured 24 h later. In these investigations, the effects of emu oil on the inflammatory response were evaluated by injecting 50 μl of the Makin emu oil intraperitoneally, three hours prior to the antigen challenge. The data presented in FIG. 5 show that mice which had been pretreated with emu oil developed a significantly depressed DTH response, thus showing that emu oil has anti-inflammatory activity.

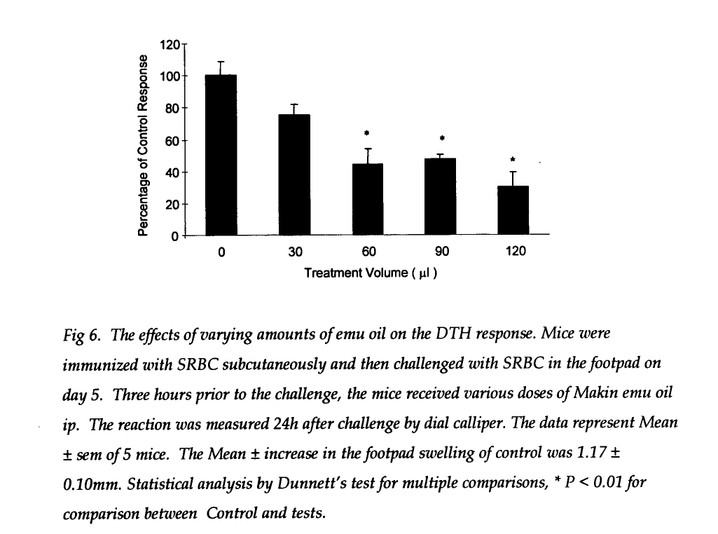

This activity of emu oil was found to be proportionately decreased as the amount of emu oil injected was decreased (FIG. 6). Thus, when 120 μl was injected, there was approximately 70% suppression of the DTH response, compared to 25% with 30 μl emu oil.

Several experiments were conducted to examine the reproducibility of the effects of Makin emu oil on DTH inflammation. The oil was administered in 50 μl ip. The results presented in Table 10 show that, in all cases, the emu oil was active in suppressing the inflammatory response.

TABLE 10
Summary of experiments examining the effects of Makin
emu oil on the DTH response

| Experimental | % inhibition of DTH response |
|---|---|
| Number | (Mean ± sem) |
| 1 | 42.9 |
| 2 | 46.7 |
| 3 | 38.8 |
| 4 | 43.5 |
| 5 | 25.2 |
| 6 | 56.0 |
| Mean ± sem | 42.2 ± 4.1 |

Mice were immunised subcutaneously with SRBC and 5 days later challenged with SRBC subcutaneously in the hind footpad. Three hours prior to challenge, the mice were treated with 50 μl of emu oil ip. The DTH reaction was assessed by measuring the thickness of footpad swelling. Five mice per group were used in each experiment.

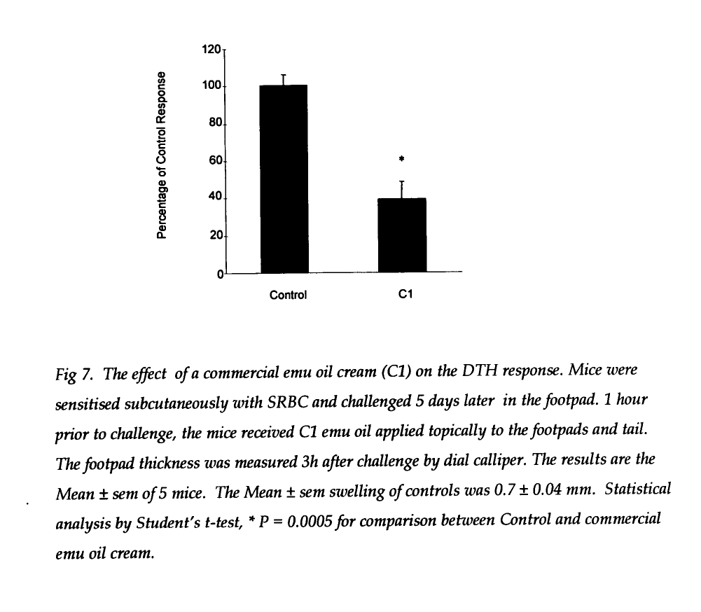

A commercial source of emu oil cream from Emu Oil Therapies (EOT) designated as C1 was tested. The ointment is for topical application and contains small amounts of eucalyptus and lavender oils. The cream was applied to the footpads of mice 1 h prior to challenge with SRBC. The results presented in FIG. 7 show that C1 was highly immunosuppressive, causing a 60% reduction in footpad swelling.

12.2 Comparison of the Anti-Inflammatory Properties of Different Emu Oil Preparations

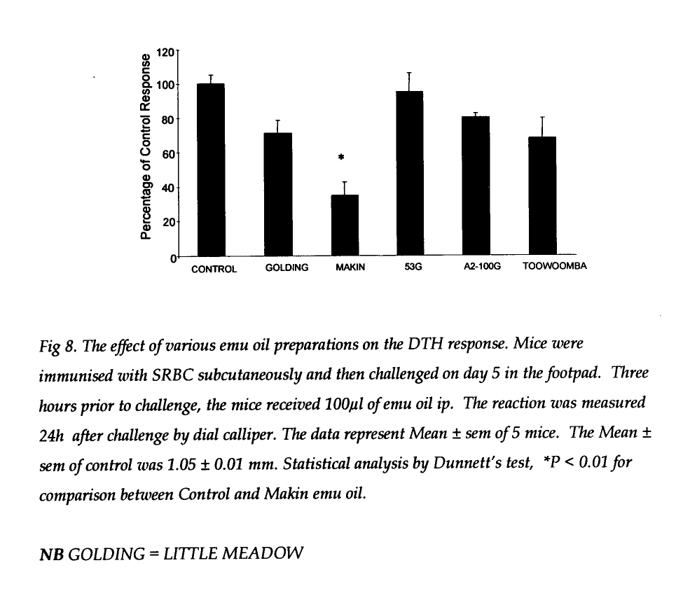

The various emu oil preparations which had undergone chemical analyses were also compared in their ability to reduce the inflammatory response. Groups of mice were sensitised with SRBC and, 3 h prior to antigen challenge, received one type of emu oil intraperitoneally. It is evident from the results presented in FIG. 8 that Makin emu oil was the most effective. The others showed very poor anti-inflammatory activity.

12.3 Comparison of the Pre and Post Antigen Challenge Treatment with Emu Oil

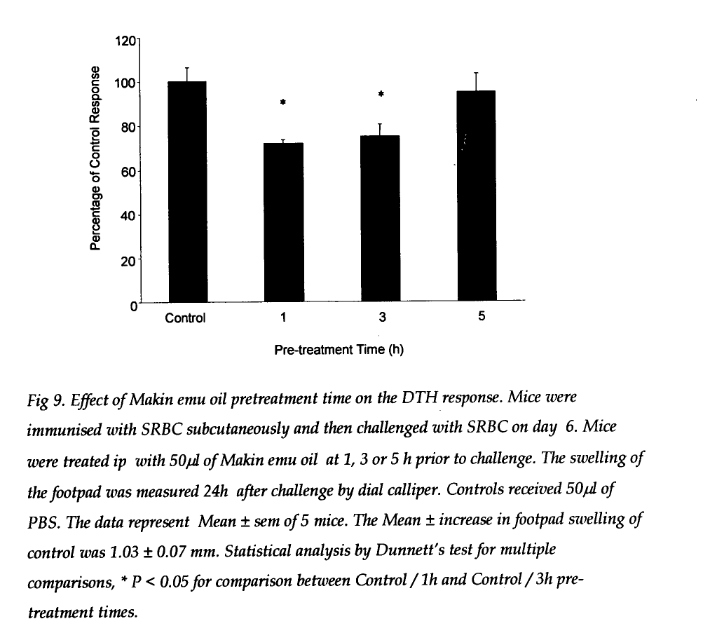

The utility of a substance to treat an inflammatory reaction can be assessed on its ability to stop inflammation even after it has been elicited. This was examined for emu oil using the DTH model. In initial studies, experiments were conducted in which the emu oil pretreatment time was varied from 1 to 5 h prior to challenge. Thus, SRBC primed mice were pretreated at 1,3 and 5 h prior to SRBC challenge with 50 μl of Makin emu oil intraperitoneally. The results showed that the oil was most effective if given 1 h prior to challenge (FIG. 9).

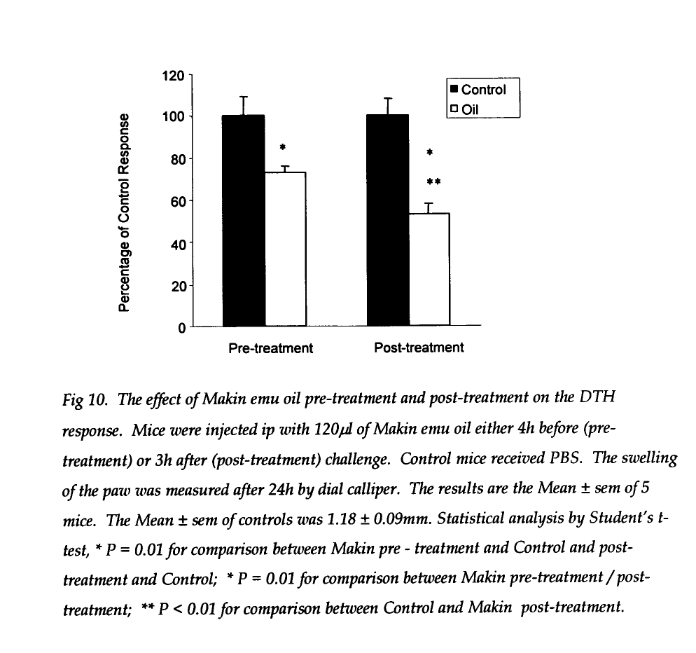

In further experiments, the effects of delaying treatment of mice with emu oil until 3 h after challenge with SRBC on the development of the DTH reactions were examined. Investigations were set up to compare the effects of 3 h pre-treatment versus 3 h post-treatment in relation to antigen challenge. The results showed that Makin emu oil was just as effective if the treatment were delayed and, in fact, delayed treatment was significantly more suppressive than treatment given prior to challenge (FIG. 10).

12.4 Effects of Emu Oil on Acute Inflammation

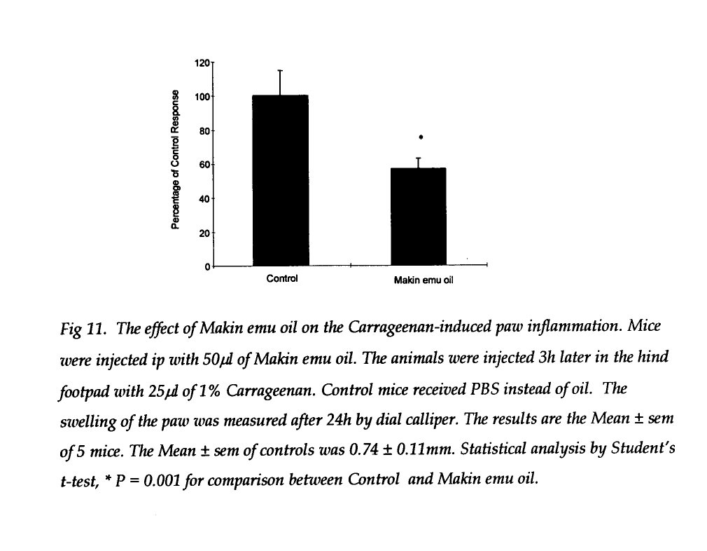

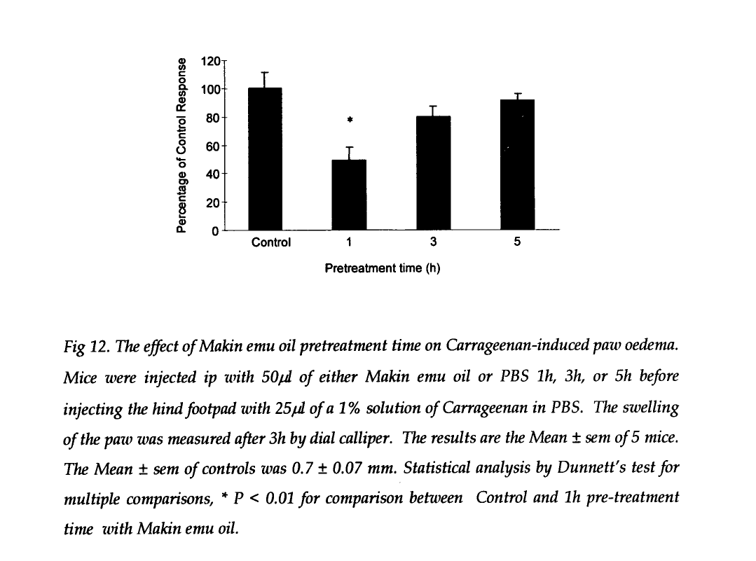

Acute inflammation is dominated by neutrophils rather than T lymphocytes and macrophages, although the latter two cell types are also likely to have a role. This can be tested using an established model of carrageenan induced inflammatory responses. This model was used to examine the effects of emu oil on acute inflammation. Mice were treated intraperitoneally with Makin emu oil 3 h prior to receiving carrageenan into the hind footpad. The swelling was then measured 24 h after the injection of carrageenan. The data showed that the oil was quite effective in depressing the carrageenan-induced inflammatory response (FIG. 11). As per DTH reaction, comparison of pretreatment of mice for 1 h, 3 h, 5 h showed 1 h to be most effective (FIG. 12).

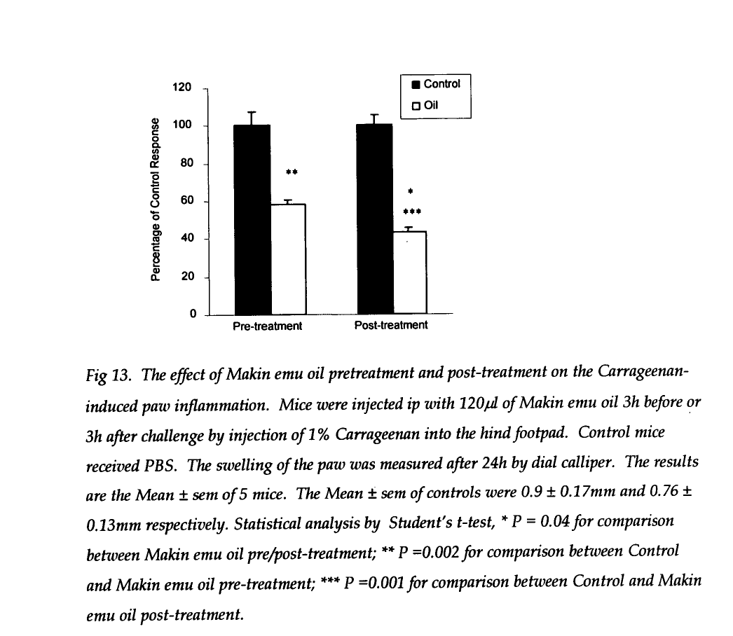

Examination of emu oil post-treatment with respect to acute inflammation and carrageenan-induced inflammation showed that the delayed treatment was just as effective with this model in inhibiting inflammation (FIG. 13). As with chronic inflammation, a greater degree of suppression of inflammation was seen.

12.5 Effect of Rendering Temperature on Emu Oil Chemical Composition and Anti-Inflammatory Activity

Makin emu fat (EF) was subjected to heating at 40° C. for 2 h, the oil removed and the remaining fat subjected to heating at 60° C. for 2 h. After collection of the oil, the fat was heated at 80° C. and the oil produced under this temperature collected.

The oils prepared under the three different rendering conditions were analysed by GC. The results are presented in Table 11.

TABLE 11
GC Analyses of fatty acids in emu oils prepared
at different rendering temperatures

| Fatty Acid | makin | EO1-40C. | EO1-60C. | EO1-80C. |
|---|---|---|---|---|
| 8:0 | | | | |
| 9:0 | | | | |
| 10:0 | | | | |
| 11:0 | | | | |
| 12:0 | 0.03 | 0.03 | 0.30 | 0.03 |
| 13:0 | | | | |
| 14:0 | 0.42 | 0.39 | 0.37 | 0.40 |
| 15:0 | 0.07 | 0.03 | 0.03 | 0.03 |
| dma 16:0 | | | | |
| 16:0 | 20.54 | 26.65 | 26.71 | 27.13 |
| 17:0 | 0.22 | 0.08 | 0.09 | 0.09 |
| dma 18:0 | | | | |
| 18:0 | 11.14 | 8.12 | 8.49 | 7.90 |
| 20:0 | 0.21 | 0.10 | 0.10 | 0.09 |
| 22:0 | 0.03 | | | |
| 24:0 | | | | |
| Total Sets | 32.67 | 35.39 | 35.82 | 35.87 |
| Trans 16:1 | 0.03 | | | |
| Trans 18:1ω9 | 0.32 | 0.23 | 0.23 | 0.23 |
| Trans 18:1ω7 | 0.19 | | | |
| Trans 18:2 | 0.04 | | | |
| Totals Trans | 0.68 | 0.23 | 0.23 | 0.23 |
| 11:1 | | | | |
| 12:1 | | | | |
| 13:1 | | | | |
| 14:1 | 0.09 | 0.14 | 0.13 | 0.16 |
| 15:1 | | | | |
| 16:1ω9 | 0.15 | 0.10 | 0.10 | 0.11 |
| 16:1ω7 | 2.95 | 5.71 | 5.51 | 6.05 |
| 17:1 | | | | |
| 18:1ω9 | 47.88 | 46.37 | 48.31 | 48.64 |
| 18:1ω7 | 2.68 | 2.71 | 2.72 | 1.84 |
| 19:1 | 0.05 | | | |
| 20:1ω11 | 0.06 | 0.06 | 0.06 | 0.06 |
| 20:1ω9 | 0.41 | 0.25 | 0.25 | 0.23 |
| 22:1ω11 | | | | |
| 22:1ω9 | 0.02 | | | |
| 24:1ω9 | | | | |
| Total Monos | 53.41 | 55.32 | 55.06 | 54.90 |
| 18:2ω9 | 0.02 | | | |
| 20:2ω9 | 0.03 | | | |
| 20:3ω9 | 0.03 | 0.02 | 0.03 | 0.03 |
| Total ω9 | 49.26 | 46.75 | 48.89 | 46.00 |
| Total ω7 | 4.78 | 8.42 | 8.22 | 8.73 |
| 9,11 18:2 cLA | 0.06 | 0.05 | 0.05 | 0.05 |
| 10,12 18:2 cLA | | | | |
| 18:2ω6 | 11.95 | 8.19 | 7.98 | 8.30 |
| 18:3ω6 | 0.04 | | 0.02 | |
| 20:2ω6 | 0.10 | 0.06 | 0.06 | 0.06 |
| 20:3ω6 | 0.02 | | | |
| 20:4ω6 | 0.09 | 0.04 | 0.04 | 0.05 |
| 22:2ω6 | | | | |
| 22:4ω6 | 0.04 | | | |
| 22:5ω6 | | | | |
| Total ω6 | 12.24 | 8.28 | 8.10 | 8.41 |
| 16:2ω3 | 0.08 | | 0.02 | |
| 18:3ω3 | 0.82 | 0.67 | 0.65 | 0.70 |
| 18:4ω3 | | | | |
| 20:3ω3 | | | | |
| 20:5ω3 | | | | |
| 22:5ω3 | 0.04 | | 0.02 | |
| 22:6ω3 | | | | |
| Total ω3 | 0.94 | 0.67 | 0.89 | 0.70 |

The results showed that the three preparations were almost identical in terms of composition of the major and minor fatty acids. When compared to other emu oil preparations, the composition of fat was similar.

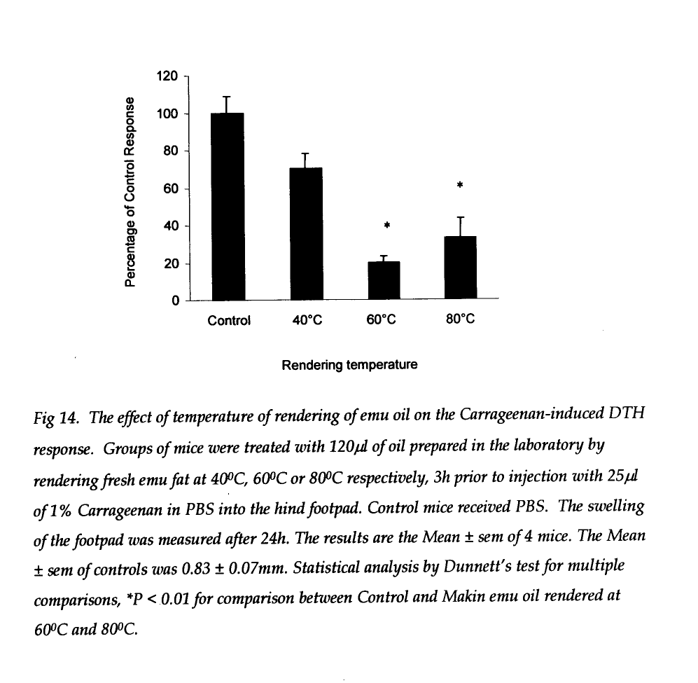

The three oils were then tested for their effects on the carrageenan-induced inflammatory response. Mice were pretreated for 3 h and 120 μl of each of the emu oil preparations (40° C., 60° C. or 80° C.) and then treated with carrageenan in the hind paw. The result showed that, while all three inhibited the inflammatory reaction, 60° C. rendering produced the most effective oil followed by 80° C. (FIG. 14). While the rendering temperature effects were also seen in the DTH reaction, it was the 80° C. and 100° C. oil preparations which were most anti-inflammatory (FIG. 15).

12.6 Activity of the Ethanol Soluble Fraction of Emu Oil

The ethanol soluble component of Makin emu oil was prepared and examined for anti-inflammatory properties by using several in vitro parameters of inflammation. The ethanol soluble fraction was tested for ability to depress T lymphocyte, macrophage and neutrophil responses.

12.6.1 T Lymphocyte Responses

Makin emu oil was subjected to solubility in ethanol. This ethanol soluble oil fraction was then tested for ability to depress proliferation of mitogen stimulated human lymphocytes. The mononuclear cells were isolated from peripheral blood and pretreated for 30 min with dilutions of the fraction and then challenged with phytohaemagglutinin (PHA). Proliferation of lymphocytes was measured after 48 hours using ³H-TdR incorporation as a marker for DNA synthesis.

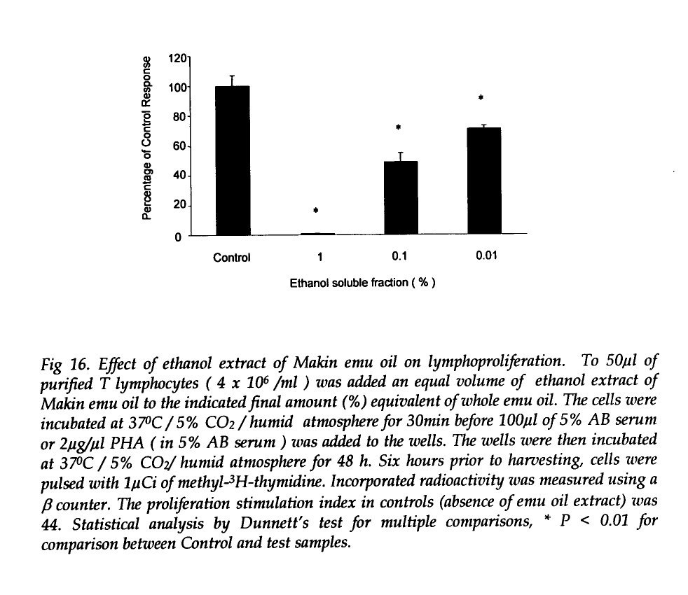

Lymphocytes pretreated with the ethanol soluble fraction of emu oil showed marked inhibition of PHA-induced lymphoproliferation (FIG. 16). This aspect has been repeated several times and similar results were obtained reproducibly. Table 12 shows the results from a number of experiments which have examined the effect of ethanol extracts of Makin emu oil on lymphoproliferation. Using this assay system, the ethanol fractions from oils rendered at 40° C., 60° C. and 80° C. were tested. Interestingly, 60° C. and 80° C. oils were more active than 40° C. (FIG. 17).

TABLE 12
Summary of experiments examining the effects of various ethanol
extractions of Makin emu oil on the lymphoproliferation response
in human T lymphocytes stimulated with PHA. A volume of 50 μl
of purified T lymphocytes (4 × 10⁶/ml) was placed into a
U-bottom well and an equal volume of ethanol or ethanol extract
of Makin emu oil (final of 1% whole emu oil equivalent) was
added to the wells. The cells were incubated at 37° C./5%
CO₂/humid atmosphere for 30 min before 100 μl of 5%
AB serum or 2 μg/μl PHA (in 5% AB serum) was added to the
wells. The wells were then incubated at 37° C./5% CO₂/
humid atmosphere for 48 hours. Six hours prior to harvesting,
the cells were pulsed with 1 μCi of methyl-³H-thymidine.
Incorporated radioactivity was measured using a β counter.

| Experimental | % Inhibition of |
|---|---|
| Number | Lymphoproliferative Response |
| 1. | 84.3 ± 1.5 |
| 2. | 84.2 ± 5.5 |
| 3. | 85.0 ± 8.5 |
| 4. | 99.9 ± 0.14 |
| 5. | 99.75 ± 0.045 |
| Mean ± sem | 90.63 ± 3.76 |

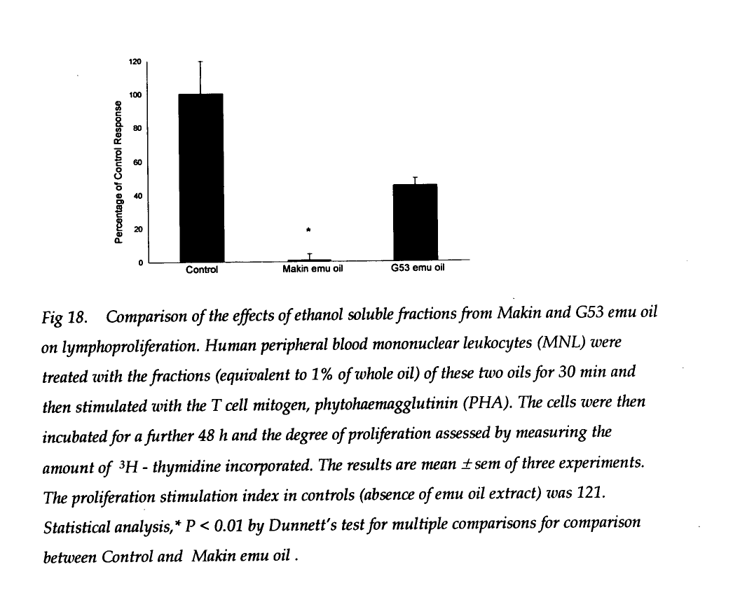

Considering that Makin emu oil was found to be highly active in inhibiting DTH in comparison to G53 emu oil, the ethanol fractions from the two oil preparations were compared in their abilities to inhibit T lymphocyte proliferation induced by PHA. The data presented in FIG. 18 show that, while Makin emu oil caused >90% inhibition of the T lymphocyte response, G53 emu oil produced only 50% inhibition of this response.

12.6.2 Monocyte Function

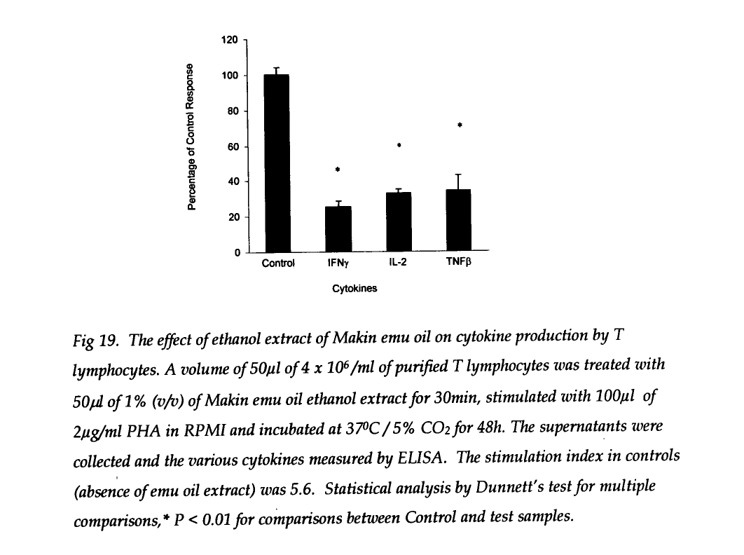

Further experiments examined the effect of emu oil on cytokine production by T lymphocytes. As per lymphocyte proliferation assays, the mononuclear leukocyte fraction was pretreated with the Makin emu oil ethanol fraction and then stimulated with PHA. After 48 h incubation, the supernatants were assessed for levels of the cytokines, IFN-γ, TNF-β and IL-2 (FIG. 19).

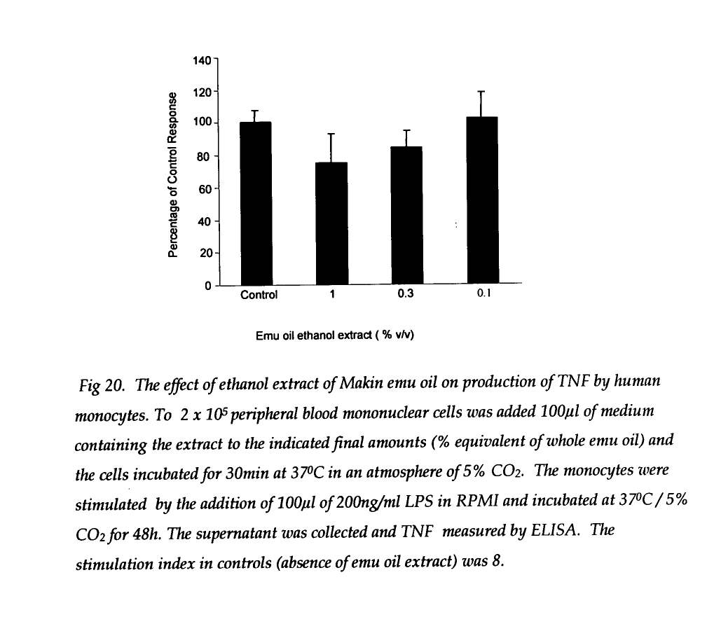

The results showed that production of these cytokines, and in particular IFN-γ, was inhibited. Monocytes prepared as the adherent fraction of mononuclear leukocytes were pretreated with Makin emu oil ethanol fraction and then stimulated with bacterial lipopolysaccharide (LPS). The effect on TNF-α production was assessed by measuring the cytokine in the cultured treated or untreated monocytes. The results showed that Makin ethanol fraction of emu oil was a poor inhibitor of LPS-induced cytokine production (FIG. 20).

12.6.3 Neutrophil Adherence

Since neutrophils are the main proponents of acute inflammation, investigations were conducted as to whether the ethanol soluble emu oil fraction affected neutrophil functional responses essential for neutrophil tissue influx and whether accumulation of neutrophils at inflammatory sites requires the adhesion of neutrophils to the endothelium of blood vessels. This adhesion can be promoted by upregulating integrins on the neutrophil surface, as well as adhesion molecules on the endothelial tissue.

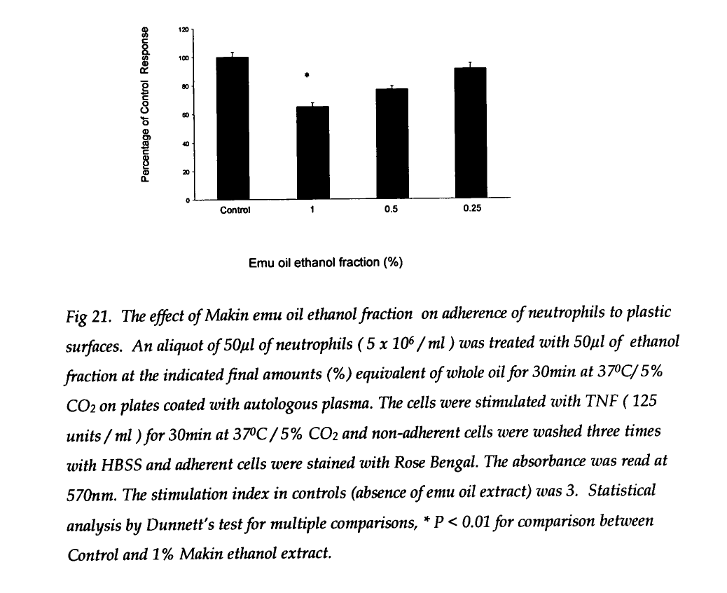

In the first set of investigations, neutrophils were exposed to Makin emu oil ethanol fraction and then stimulated with phorbol myristate acetate (PMA). The results showed that the PMA-induced upregulation of neutrophil adhesion to plastic surfaces was depressed by treatment with this fraction of oil (FIG. 21).

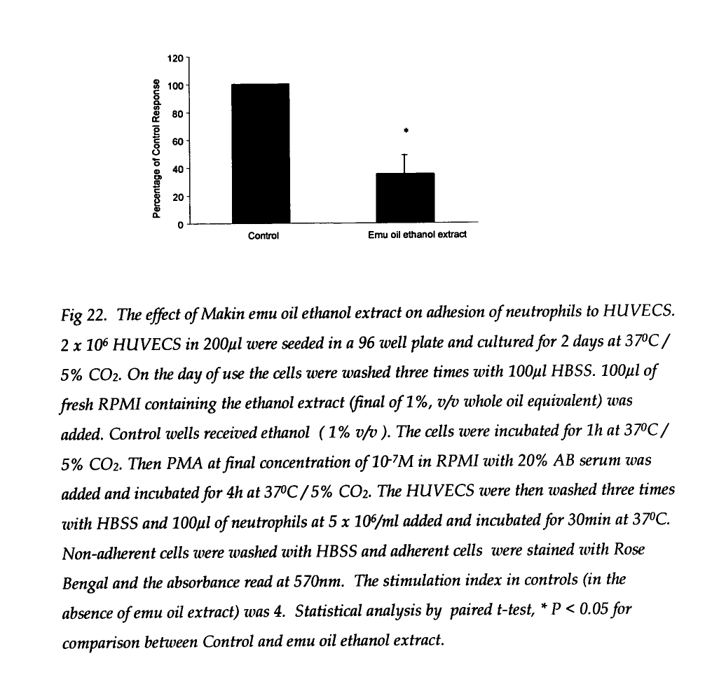

In the second set of investigations, human umbilical vein endothelial cells were exposed to the Makin emu oil ethanol fraction. The cells were washed and then stimulated with tumor necrosis factor (TNF) to upregulate the adhesion molecules. Fresh neutrophils were added to the endothelial cell monolayers and the degree of neutrophil adherence was quantified. The (TNF) stimulated endothelial cells showed enhanced neutrophil adhesion and this was significantly reduced in endothelial cell cultures which had been pretreated with the emu oil (FIG. 22).

12.6.4 Neutrophil Chemotaxis

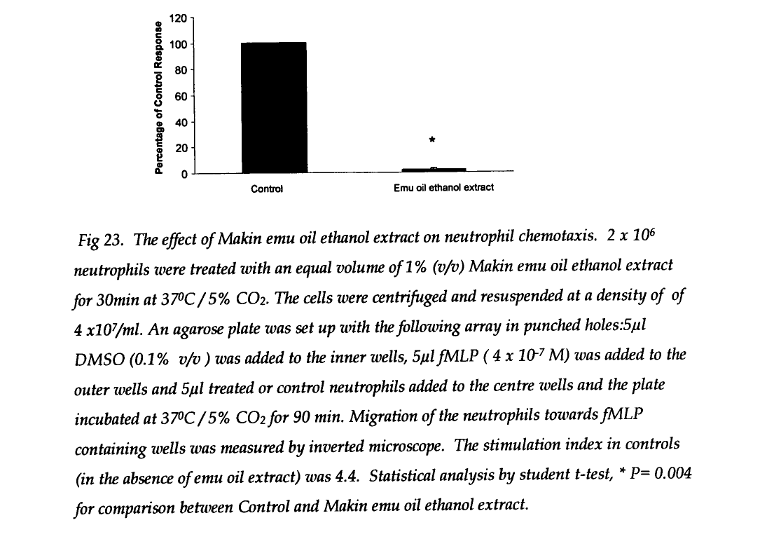

The ability of neutrophils to move into infection sites is dependent on their chemotactic response. In this investigation, the neutrophil chemotaxis response was quantified by measuring the degree of movement of neutrophils towards a chemotactic agent, the tripeptide FMLP. The data presented in FIG. 23 show that neutrophils, which had been pretreated with Makin emu oil ethanol fraction, showed a poor chemotactic response.

12.7 Further Characterisation of the Anti-T Cell Activity of Emu Oil

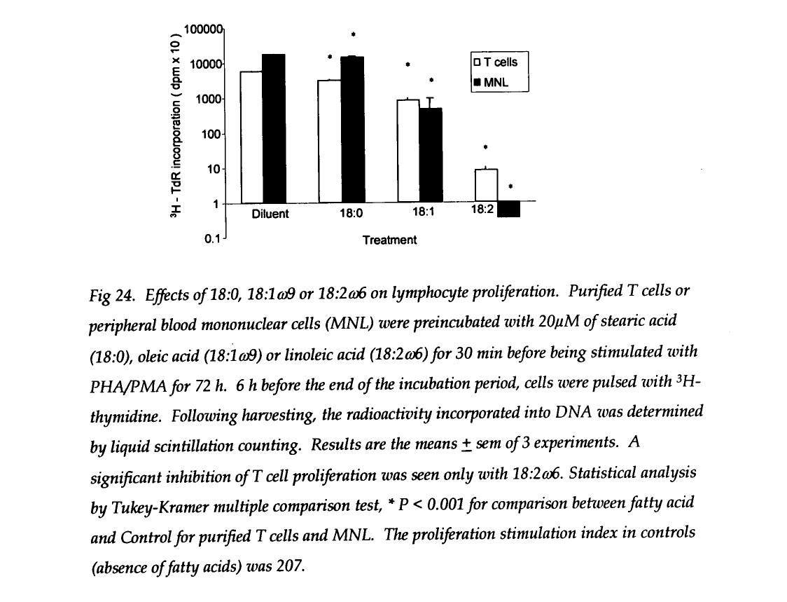

Preliminary studies have also shown that some of the unsaturated fatty acids found in emu oil inhibit T lymphocyte and mononuclear cell responses. Thus, our results show that 18:2ω6 is strongly inhibitory compared with 18:1ω9, 18:0 and 18:2 (FIG. 24).

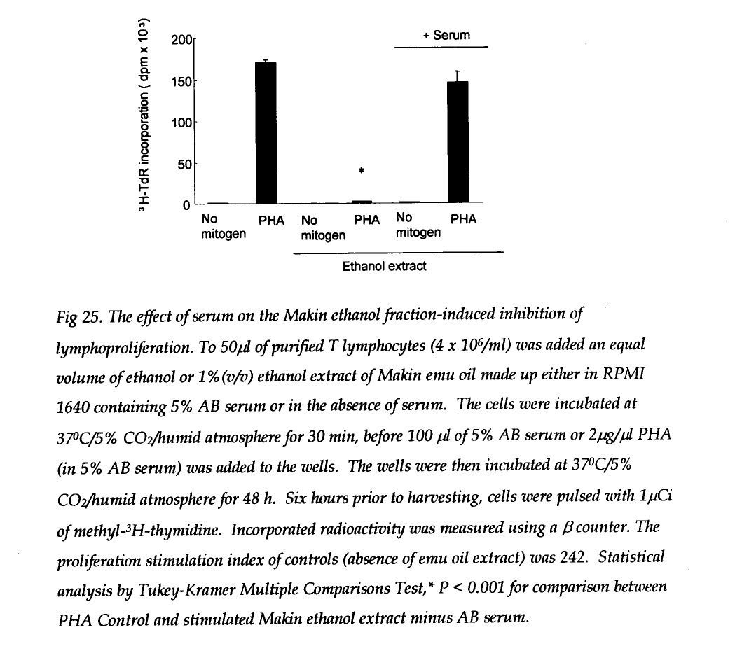

Since long chain fatty acids such as 18:2ω6 are suspected to be responsible for the anti T cell effects, it was interesting to see if the fatty acid binding proteins in serum could prevent the activity present within the ethanol fraction. The lymphocytes were pretreated with the Makin emu oil ethanol fraction in the presence and absence of 5% human blood group AB serum and then stimulated with PHA. The data in FIG. 25 show that serum could prevent the inhibitory effects of the emu oil ethanol fraction on T lymphocytes.

Chemical analysis of the ethanol fraction of Makin emu oil by GC showed that the fatty acids were present in similar proportions to the whole oil (Table 13). However, there was a small increase in 18:2ω6.

TABLE 13
GC fatty acid analyses of ethanol extract of Makin emu oil

| Fatty Acid | whole oil | ethanol soluble extract |
|---|---|---|
| 8:0 | | |
| 9:0 | | |
| 10:0 | | |
| 11:0 | | |
| 12:0 | 0.03 | 0.08 |
| 13:0 | | |
| 14:0 | 0.42 | 0.59 |
| 15:0 | 0.07 | 0.08 |
| dma 16:0 | | |
| 16:0 | 20.54 | 18.91 |
| 17:0 | 0.22 | 0.19 |
| dma 18:0 | | |
| 18:0 | 11.14 | 7.95 |
| 20:0 | 0.21 | 0.18 |
| 22:0 | 0.03 | |
| 24:0 | | |
| Total Sets | 32.67 | 27.99 |
| Trans 16:1 | 0.03 | |
| Trans 18:1w9 | 0.32 | 0.30 |
| Trans 18:1w7 | 0.19 | 0.21 |
| Trans 18:2 | 0.04 | |
| Totals Trans | 0.58 | 0.51 |
| 11:1 | | |
| 12:1 | | |
| 13:1 | | |
| 14:1 | 0.09 | 0.16 |
| 15:1 | | |
| 16:1w9 | 0.15 | 0.18 |
| 16:1w7 | 2.95 | 4.06 |
| 17:1 | | |
| 18:1w9 | 47.88 | 48.57 |
| 18:1w7 | 1.84 | 1.93 |
| 19:1 | 0.05 | |
| 20:1w11 | 0.06 | |
| 20:1w9 | 0.41 | 0.38 |
| 22:1w11 | | |
| 22:1w9 | 0.02 | 0.14 |
| 24:1w9 | | |
| Total Monos | 53.41 | 55.40 |
| 18:2w9 | 0.02 | |
| 20:2w9 | 0.03 | |
| 20:3w9 | 0.03 | |
| Total w9 | 48.53 | 49.26 |
| Total w7 | 4.78 | 5.99 |
| 9,11 18:2 cLA | 0.06 | 0.05 |
| 10,12 18:2 cLA | | |
| 18:2w6 | 11.95 | 14.69 |
| 18:3w6 | 0.04 | 0.04 |
| 20:2w6 | 0.10 | 0.11 |
| 20:3w6 | 0.02 | |
| 20:4w6 | 0.09 | 0.21 |
| 22:2w6 | | |
| 22:4w6 | 0.04 | |
| 22:5w6 | | |
| Total w6 | 12.24 | 15.01 |
| 16:2w3 | 0.08 | |
| 18:3w3 | 0.82 | 1.04 |
| 18:4w3 | | |
| 20:3w3 | | |
| 20:5w3 | | |
| 22:5w3 | 0.04 | |
| 22:6w3 | | |
| Total w3 | 0.94 | 1.04 |

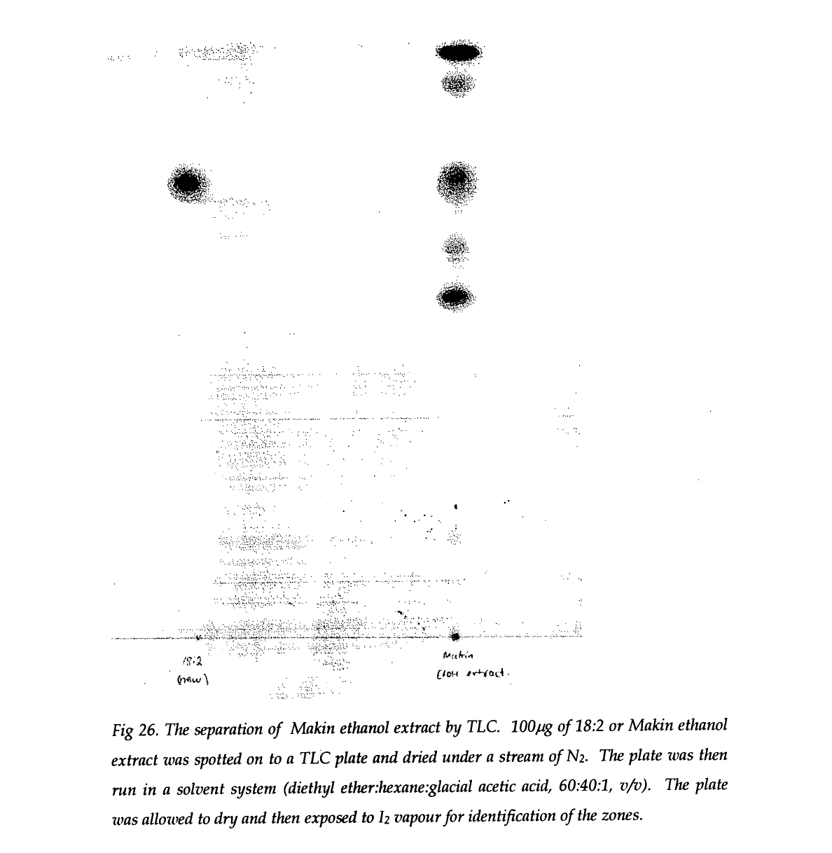

The ethanol soluble emu oil fraction was also subjected to TLC (analytical). This revealed seven bands (FIG. 26). Interestingly, band 3 corresponded to 18:2ω6. A preparative run was also conducted and this is shown in FIG. 27, revealing 8 fractions. These fractions were then tested for the ability to inhibit lymphocyte proliferation. The results showed that the major activity was associated with fractions 3,4 and 6, equalling fraction 3 (FIG. 28). The other fractions had much less activity. Interestingly, fraction 3 corresponds to 18:2ω6 mobility.

12.8 Anti-Inflammatory Properties of Emu Oil Triglyceride Fraction

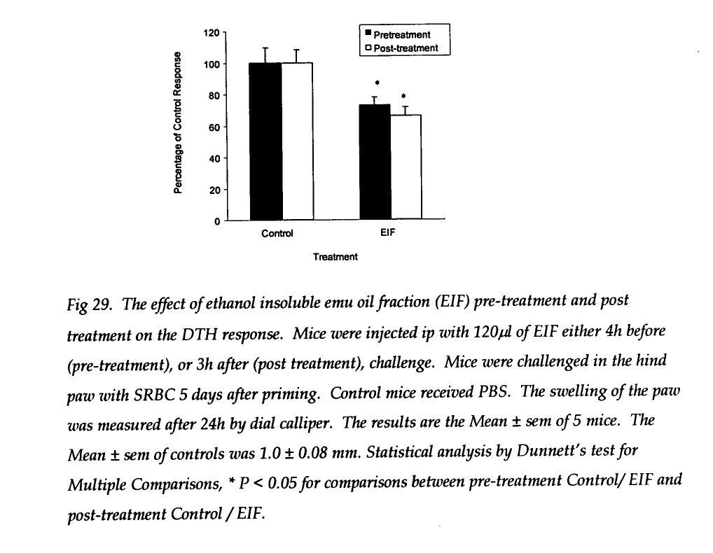

The ethanol insoluble fraction contains primarily the triglyceride component of the oil. This was tested for inhibiting activity on the DTH reaction. In these experiments, mice were treated with the triglyceride fraction of emu oil either 3 h prior to antigen challenge or 3 h post-challenge. The DTH response was significantly reduced to a similar extent as the whole oil when the triglyceride fraction was applied either prior to or post antigen challenge (FIG. 29).

### REFERENCES

1. Kresina, T. F. (ed) Immune Modulating Agents. Marcel Dekker, New York, pp 1-557, (1997).
2. Craig-Schmidt, M. Emu: premier oil bird. IFORM 8, 246-252 (1997).
3. Bennett, G. Observations principally on the Animal and Vegetable Products of New South Wales; Gatherings of a Naturalist in Australia. London. John Van Voorst, Paternoster Row; 1860.
4. Snowden, J. M. and Whitehouse, M. W. Anti-inflammatory activity of emu oils in rats. Inflammopharmacology, 5,127-132 (1997).
5. Lopez, A., Sims, D. E., Ablett, R. F., Skinner, R. E., Leger, L. W., Lariviere, C. M., Jamieson, L. A., Martinez-Burnes, J. A. and Zawadzka, G. G., Effect of emu oil on auricular inflammation induced with croton oil in mice. Am. J. Vet. Res. 60(12):1558-1561 (1999).
6. Fein, E., Caputo, J. and Nagal, K. Therapeutic uses of emu oil. U.S. Pat. No. 5,472,713, issued 1995.
7. Whitehouse M. W., Turner A. G., Davis C. K. C. and Roberts M. S. Emu oil(s): a source of non-toxic transdermal anti-inflammatory agents in aboriginal medicine. Inflammopharmacology, 6, 1-8 (1997).
8. Ferrante, A, Rowan-Kelly, B. & Thong, Y. H. Suppression of the immunological responses in mice by treatment with amphotericin B. Clin. & Exp. Immunol. 38, 70-76 (1979).
9. Costabile, M., Hii, C. S. T., Robinson, B. S., Rathjen, D. A., Pitt, M. J., Easton, C., Miller, R. C., Poulos, A., Murray, A. W., and Ferrante, A. A novel long chain polyunsaturated fatty acid, β-oxa 21:3n-3, inhibits T lymphocyte proliferation, cytokine production, delayed type hypersensitivity, carrageenan-induced paw oedema reaction and selectively targets intracellular signals. J. Immunol. 167:3980-3987 (2001).
10. Fletcher, D., Kayser, V., & Guilbaud, G. Influence of timing of administration on the analgesic effect of bupivacaine infiltration in carrageenan-injected rats. Anesthesiology 84, 1129-1137 (1996).
11. Ferrante, A. & Thong, Y. H. Optimal conditions for the simultaneous purification of mononuclear and polymorphonuclear leukocytes from human blood by the hypaque-ficoll method. J. Immun. Methods 36, 109-117 (1980).
12. Ferrante, A., Rowan-Kelly, B. & Thong, Y. H. Inhibition of mitogen induced human lymphocyte responsiveness by polymixin antibiotics. Int. Arch. Allerg. & Appl. Immunol. 69, 279-281 (1982).
13. Staugas, R. E. M., Harvey, D. P., Ferrante, A., Nandoskar, M. and Allison, A. C. Induction of tumour necrosis factor and interleukin-1 by Pseudomonas aeruginosa and exotoxin A-induced suppression of lymphoproliferation, TNF, lymphotoxin, interferon gamma and interleukin-1 in human leukocytes. Infect. Immun. 60, 3162-3168 (1992).
14. Bates, E. J., Ferrante, A., Robinson, B., Smithers, L. and Poulos, A. Effect of fatty acid structure on neutrophil adhesion, degranulation and damage to endothelial cells. Atherosclerosis. 116, 247-259 (1995).
15. Huang, Z. H., Bates, E. J., Ferrante, J. V., Poulos, A., Robinson, B. S. and Ferrante, A. Inhibition of stimuli-induced endothelial cell intercellular adhesion molecule-1, E-selectin and vascular cellular adhesion molecule-1 expression by arachidonic acid and its hydroxy- and hydroperoxy-derivatives. Circulation Research. 80, 149-158 (1997).
16. Ferrante, A., Goh, D. H. B., Harvey, D. P., Robinson, B. S., Hii, C. S. T., Bates, E. J., Hardy, S. J., Johnson, D. W. and Poulos, A. Neutrophil migration inhibitory properties of polyunsaturated fatty acids: the role of fatty acid structure, metabolism and possible second messenger systems. J. Clin. Invest. 93, 1063-1070 (1994).
17. Visioli, F. and Galli, C. The effects of minor constituents of olive oil on cardiovascular disease: new findings. Nutrition Reviews 56, 142-147 (1998).

1-31. (canceled)

32. An assay system for grading a substance so as to assess, in a standardized manner, its anti-inflammatory activity, said assay system comprising: (i) injection of a suitable antigen, into an appropriate body part of a mammal; (ii) either injection of a predetermined amount of said test substance into the same body part, or topical application to said mammal of a predetermined amount of said substance; (iii) measurement of the degree to which swelling which would otherwise result from injection of said antigen is reduced or alleviated; and (iv) comparing the activity of said test substance, as measured in step (iii), against the activity of a standard compound having known anti-inflammatory characteristics, the activity of said standard compound having been measured by this same assay system of steps (i) to (iii), and having been used to generate a grading system to compare the efficacy of various of the assessed substances.

33. An assay system according to claim 32, wherein said substance is selected from the group consisting of oils, fats, organic solvent extracts of oils and fats, preparations comprising oils, preparations comprising fats, biologically active components of oils, and biologically active components of fats.

34. An assay system according to claim 33, wherein said substance is selected from the group consisting of animal oils and plant oils.

35. An assay system according to claim 34 wherein the oil is selected from the group consisting of tea tree oil, flaxseed oil, linseed oil, borage oil and evening primrose oil; fish oils; and algal, microbial and fungi oils.

36. An assay system according to claim 33, wherein said substance is emu oil or an ethanol extract of emu oil.

37. An assay system according to claim 32 wherein, in step (i), said antigen is injected intraperitoneally or into a footpad or ear of said mammal.

38. An assay system according to claim 32, wherein said antigen is Carrageenan or sheep red blood cells.

39. An assay system according to claim 32 wherein, in step (ii), said substance is injected intraperitoneally or applied topically.

40. An assay system according to claim 32 wherein steps (i) to (iv) are repeated, using serially reducing amounts of said substance.

41. An assay system according to claim 40, wherein said substance is serially diluted in ethanol.

42. An assay system for grading a substance so as to assess, in a standardized manner, its anti-inflammatory activity, said assay system comprising:
(i) measurement of the activity of an in vitro preparation of T-cells, macrophages or neutrophils, or a cell line derived therefrom;
(ii) addition of said substance to said preparation of T-cells, macrophages or neutrophils, or said cell line derived therefrom;
(iii) measurement of the change in activity of said preparation of T-cells, macrophages or neutrophils, or said cell line derived therefrom, following addition of said substance in step (ii); and
(iv) comparing the change in activity, as measured in step (iii), for said substance against the change in activity for a standard compound having known anti-inflammatory characteristics, the change in activity for the standard compound having been measured by this same assay system of steps (i) to (iii), and having been used to generate a grading system to compare the efficacy of various of the assessed substances.

43. An assay system according to claim 42, wherein said substance is selected from the group consisting of oils, fats, organic solvent extracts of oils and fats, preparations comprising oils, preparations comprising fats, biologically active components of oils, and biologically active components of fats.

44. An assay system according to claim 43, wherein said substance is selected from the group consisting of animal oils and plant oils.

45. An assay system according to claim 44 wherein said oil is selected from the group consisting of tea tree oil, flaxseed oil, linseed oil, borage oil and evening primrose oil; fish oils; and algal, microbial and fungi oils.

46. An assay system according to claim 43, wherein said substance is emu oil or an ethanol extract of emu oil.

47. An assay system according to claim 42, wherein said preparation is a preparation of T lymphocytes and said activity is lymphoproliferation.

48. An assay system according to claim 42, wherein said preparation is a preparation of T lymphocytes and said activity is production of cytokines.

49. An assay system according to claim 48, wherein said cytokines are selected from the group consisting of interleukin-2, tumor necrosis factors and interferon-γ.

50. An assay system according to claim 42, wherein said preparation is a preparation of neutrophils and said activity is chemotaxis.

51. An assay system according to claim 42, wherein said preparation is a preparation of neutrophils and said activity is adherence to endothelial cells.

52. An assay system according to claim 42, wherein steps (i) to (iv) are repeated, using serially reducing amounts of said substance.

53. An assay system according to claim 52, wherein said substance is serially diluted in ethanol.

54. A method of preparing a therapeutically active emu oil, comprising the heating of emu oil or tissue which contains emu oil to a temperature of at least 40° C. in order to obtain the active components of the oil or tissue.

55. The method of claim 54, wherein the heating temperature is in the range of about 40° to 100° C.

56. The method of claim 54, wherein the heating temperature is in the range of about 60° to 80° C.

57. The method of claim 54, wherein said temperature is about 60° C.

58. The method of claim 54, wherein said temperature is about 80° C.

59. The method of claim 54, wherein the heating temperature is about 100° C.

60. A method of preparing a therapeutically active fraction from emu oil or emu oil containing tissue, comprising extracting said oil or tissue with an organic solvent to obtain the active components of the oil or tissue.

61. The method of claim 60, wherein the organic solvent is an alcohol.

62. The method of claim 61, wherein the alcohol is ethanol.
